SEQUENCE LISTING (32 sequences)

SEQ ID NO: 1 - RNA, 554 nt, Homo sapiens
ggguugcggagggugggccugggaggggugguggccauuuuuugucuaacccuaacugagaagggcguaggcgccgugcuuuugcuccccgcgcgcuguuuuucucgcugacuuucagcgggcggaaaagccucggccugccgccuuccaccguucauucuagagcaaacaaaaaaugucagcugcuggcccguucgccucccggggaccugcggcgggucgccugcccagcccccgaaccccgccuggagccgcggucggcccggggcuucuccggaggcacccacugccaccgcgaagaguugggcucugucagccgcgggucucucgggggcgagggcgagguucaccguuucaggccgcaggaagaggaacggagcgagucccgccgcggcgcgauucccugagcugugggacgugcacccaggacucggcucacacaugcaguucgcuuuccuguugguggggggaacgccgaucgugcgcauccgucaccccucgccggcagugggggcuugugaacccccaaaccugacugacugggccagugugcu

SEQ ID NO: 2 - DNA, 20 bp, Homo sapiens
acattttttgtttgctctag

SEQ ID NO: 3 - DNA, 30 bp, Homo sapiens
gctctagaatgaacggtggaaggcggcagg

SEQ ID NO: 4 - DNA, 14 bp, Homo sapiens
gtggaggcggcagg

SEQ ID NO: 5 - DNA, 13 bp, Homo sapiens
ggaaggcggcagg

SEQ ID NO: 6 - DNA, 13 bp, Homo sapiens
gtggaaggcggca

SEQ ID NO: 7 - DNA, 11 bp, Homo sapiens
gtggaaggcgg

SEQ ID NO: 8 - DNA, 13 bp, Homo sapiens
cggtggaaggcgg

SEQ ID NO: 9 - DNA, 13 bp, Homo sapiens
acggtggaaggcg

SEQ ID NO: 10 - DNA, 16 bp, Homo sapiens
aacggtggaaggcggc

SEQ ID NO: 11 - DNA, 18 bp, Homo sapiens
atgaacggtggaaggcgg

SEQ ID NO: 12 - DNA, 13 bp, Homo sapiens
tagggttagacaa

SEQ ID NO: 13 - DNA, 13 bp, Homo sapiens
cagttagggttag

SEQ ID NO: 14 - DNA, 12 bp, Homo sapiens
tagggttagaca

SEQ ID NO: 15 - DNA, 11 bp, Homo sapiens
tagggttagac

SEQ ID NO: 16 - DNA, 11 bp, Homo sapiens
gttagggttag

SEQ ID NO: 17 - DNA, 13 bp, Homo sapiens
gttagggttagac

SEQ ID NO: 18 - DNA, 15 bp, Homo sapiens
gttagggttagacaa

SEQ ID NO: 19 - DNA, 12 bp, Homo sapiens
cccttctcagtt

SEQ ID NO: 20 - DNA, 12 bp, Homo sapiens
cgcccttctcag

SEQ ID NO: 21 - DNA, 13 bp, Homo sapiens
taggtgtaagcaa

SEQ ID NO: 22 - DNA, 13 bp, Homo sapiens
ttgtctaacccta

SEQ ID NO: 23 - DNA, 11 bp, Homo sapiens
gttgagtgtag

SEQ ID NO: 24 - DNA, 10 bp, Homo sapiens
tttttttttt

SEQ ID NO: 25 - RNA, 11 nt, Homo sapiens
cuaacccuaac

SEQ ID NO: 26 - DNA, 18 bp, Homo sapiens
atcgcttctcggcctttt

SEQ ID NO: 27 - DNA, 24 bp, Homo sapiens
cccttacccttacccttaccctaa

SEQ ID NO: 28 - DNA, 18 bp, Homo sapiens
aatccgtcgagcagagtt

SEQ ID NO: 29 - DNA, 30 bp, Homo sapiens
gcgcggcttacccttacccttaccctaacc

SEQ ID NO: 30 - DNA, 36 bp, Homo sapiens
aatccgtcgagcagagttaaaaggccgagaagcgat

SEQ ID NO: 31 - DNA, 13 bp, Homo sapiens
taggtgtaagcaa

SEQ ID NO: 32 - DNA, 24 bp, Homo sapiens
gcgcggctaaccctaaccctaacc

(19) United States
(12) Patent Application Publication
(10) Pub. No.: US 2009/0142770 A1
(43) Pub. Date: Jun. 4, 2009

(54) Hair Follicle Pharmacodynamic Assay for Telomerase Activity

(75) Inventors: Ning F. Go, Palo Alto, CA (US); Calvin B. Harley, Murphys, CA (US); Ekaterina Bassett, Richmond, CA (US); Zhu Zhen Pirot, Redwood City, CA (US)

Correspondence Address: GERON CORPORATION;Attn. David J. Earp, 230 CONSTITUTION DRIVE, MENLO PARK, CA, 94025, US

(73) Assignee: Geron Corporation, Menlo Park, CA (US)

(21) Appl. No.: 12/327,795

(22) Filed: Dec. 3, 2008

Related U.S. Application Data
(60) Provisional application No. 60/992,274, filed on Dec. 4, 2007.

(51) Int. Cl. C12Q 1/68; C12Q 1/48

(52) U.S. Cl. 435/6; 435/15

(57) ABSTRACT

The invention is directed to methods for determining the efficacy of treatment with telomerase modulators in mammals by the analysis of the level of telomerase reverse transcriptase activity in mammalian hair follicle cells.

### CROSS REFERENCE TO RELATED APPLICATIONS

This application claims priority to U.S. Provisional Ser. No. 60/992,274, filed Dec. 4, 2007 which is incorporated by reference in its entirety.

### BACKGROUND OF THE INVENTION

1. Field of the Invention

The invention is directed to methods for determining the efficacy of treatment with telomerase modulators in mammals by the analysis of the level of telomerase reverse transcriptase activity in mammalian hair follicle cells.

2. Background

Telomeres are genetic elements located at the ends of all eukaryotic chromosomes which preserve genome stability and cell viability by preventing aberrant recombination and degradation of DNA (McClintock, 1941, Genetics vol 26, (2) pp 234-282; Muller, (1938) “The remaking of chromosomes” The collecting net, vol 13, (8) pp 181-198). In humans, the telomeric sequence is composed of 10-20 kilobases of TTAGGG repeats (Blackburn, (1991) Nature vol. 350 pp 569-573; de Lange et al., (1990) Mol. Cell Biol. Vol 10, (2) pp 518-527). There is increasing evidence that gradual loss of telomeric repeat sequences may be a timing (“clock”) mechanism limiting the number of cellular divisions in normal cells (Allsopp et al., (1992) Proc. Natl. Acad. Sci. USA, vol. 89, pp. 10114-10118; Harley et al., (1990) Nature, vol. 345, pp. 458-460; Hastie et al., (1990) Nature, vol. 346, pp. 866-868; Vaziri et al., (1993) Amer. J. Hum. Genet., vol. 52, pp. 661-667). In contrast, immortal cells are capable of maintaining a stable telomere length by upregulating or reactivating telomerase, a ribonucleoprotein enzyme that is able to add TTAGGG repeats to the ends of chromosomes (Greider and Blackburn, (1985) Cell, vol. 43, pp. 405-413; Greider and Blackburn, (1989) Nature, vol. 337, pp. 331-337; Morin, (1989) Cell, vol. 59, pp. 521-529).

Methods for detecting telomerase activity, as well as for identifying compounds that regulate or affect telomerase activity, together with methods for therapy or diagnosis of cellular senescence and immortalization by controlling or measuring telomere length and telomerase activity, have been described. See PCT patent publication No. 93/23572, U.S. Pat. Nos. 5,629,154, 5,648,215, 5,645,986, 5,695,932 and 5,489,508. Each of the foregoing patent publications is incorporated herein by reference.

For example, U.S. Pat. Nos. 5,629,154; 5,863,726 and 5,648,215 describe in detail the preparation of a cell extract using a detergent lysis method and the analysis of telomerase activity by the Telomeric Repeat Amplification Protocol (TRAP assay). The telomerase activity assays described therein involve the extension of a nucleic acid substrate by telomerase and replication of extended substrates in a primer extension reaction, such as the polymerase chain reaction (PCR).

Other telomerase extraction methods use hypotonic swelling and physical disruption of cells and telomerase activity is assayed using an oligonucleotide substrate, a radioactive deoxyribonucleoside triphosphate (dNTP) for labelling any telomerase-extended substrate, and gel electrophoresis for resolution and display of products (Morin, (1989) Cell, vol. 59, pp. 521-529). Because telomerase stalls and can release the DNA after adding the first G in the 5′-TTAGGG-3′ telomeric repeat, the characteristic pattern of products on the gel is a six nucleotide ladder of extended oligonucleotide substrates. The phase of the repeats depends on the 3′-end sequence of the substrate; telomerase recognizes where the end is in the repeat and synthesizes accordingly to yield contiguous repeat sequences.

Using the TRAP assay, telomerase activity has been detected in 85% of primary human tumors tested from a variety of tissue types (Kim et al., (1994) Science, vol. 266, pp. 2011-2015; Shay and Bacchetti, (1997) European Journal of Cancer, vol. 33, No. 5, pp. 787-791). The detection of telomerase activity in human cells almost always correlates with indefinite proliferation capability (immortalization). U.S. Pat. No. 5,648,215 describes the presence of telomerase activity in somatic cells as indicative of the presence of immortal cells, such as certain types of cancer cells, which can be used to make that determination even when the cells would be classified as non-cancerous by pathology.

Telomerase is preferentially expressed in high levels in most cancer types and typically undetectable or expressed at very low levels in normal adult tissues. Therefore inhibition of telomerase is considered a promising treatment strategy for a broad variety of solid tumor types and hematological malignancies. GRN163L is a lipidated oligonucleotide analogue with potent telomerase inhibitory activity. GRN163L is currently in clinical trials in solid tumor and hematological cancers. In addition, the up-regulation of telomerase activity is being investigated as a means for restoring function in cells undergoing premature senescence resulting from genetic defects or chronic stress due to infection.

Gene expression analysis has been used to diagnose disease conditions, determine the prognosis of disease conditions, determine the efficacy of therapeutic agents in vivo, and identify novel therapeutic agents. Current methods of obtaining tissue for gene expression analysis are invasive and include tissue biopsy. Subjects undergoing such procedures face trauma, infection and possibly death. Often it is not possible or practical to obtain repeated biopsies from the same individual to monitor the efficacy of the therapeutic agent.

All references cited herein are incorporated herein by reference in their entirety.

It would be desirable to provide a non-invasive/surrogate pharmacodynamic method of analyzing telomerase activity after treatment of a subject with a telomerase modulator.

### BRIEF SUMMARY OF THE INVENTION

The invention includes a method for measuring telomerase activity in a subject exposed to a telomerase modulator, comprising measuring the level of telomerase activity in a hair follicle cell of the subject.

In another embodiment the invention includes a method of measuring the response of a subject to a telomerase modulator, comprising measuring the level of telomerase activity in a hair follicle cell of the subject, wherein a difference in the level of telomerase activity in the hair follicle cell after exposure of the subject to the telomerase modulator as compared to the level of activity in a hair follicle cell without telomerase modulator indicates a response. In one embodiment the telomerase inhibitor is administered systemically.

In another embodiment the invention includes a method for evaluating the biological response of a subject exposed to a telomerase modulator comprising the steps of:
(a) obtaining a hair follicle from said subject;
(b) determining the telomerase activity in the hair follicle of the subject,
   wherein a difference in the level of telomerase activity in a hair follicle after exposure of the subject to the telomerase modulator as compared to the level of activity in a hair follicle without telomerase inhibitor indicates a response. In some embodiments, the method of measuring telomerase activity comprises
a) preparing a cell extract from said hair follicle
b) measuring the amount of nucleotides incorporated into elongation products by the telomerase in the cell extract isolated in step (a).

In the embodiments, the subject is a mammal. In preferred embodiments the subject is a human.

In the embodiments the hair follicle may be obtained by plucking a hair from the subject. Where the subject is a mammal, the hair plucked may be the vibrissa hair (whiskers).

In the embodiments, the hair follicle may be stained with a DNA binding label and hair follicles having live cells in the bulge region selected for measurement of telomerase activity. It has been found that analyzing only those hair follicles with live cells in the sleeve region, increases the accuracy of the analysis.

In the embodiments, the level of activity in the hair follicle cell after exposure of the subject to the telomerase modulator may be measured relative to the level of activity in hair follicle cells in the same subject prior to exposure. The number of hair follicles measured for activity is simply a size sufficient to give an accurate measurement. Accordingly from 1 to 20 hair follicles, or from 5-15 follicles can be pulled and measure at one time from a subject. Alternatively, the level of activity in the hair follicle cell of the subject exposed to the telomerase modulator may be measured relative to a standardized level of telomerase activity in the hair follicle cells of a number of subjects that have not been exposed to the telomerase modulator. The standardized level of telomerase activity can be determined from a sufficient number of individual subjects to give an accurate value. The number of individuals can vary for example, from 5 to 100, or from 10 to 50 or from 10 to 20 individuals.

In another embodiment, method the comprises the steps of:
(a) preparing a cell extract from said hair follicle;
(b) placing an aliquot of said cell extract in a reaction mixture comprising a telomerase substrate lacking a telomeric repeat sequence and a buffer in which telomerase can catalyze extension of said telomerase substrate by addition of telomeric repeat sequences;
(c) adding to said reaction mixture a primer comprising a sequence sufficiently complementary to a telomeric repeat to hybridize specifically thereto under conditions such that if an extended telomerase substrate is present in said reaction mixture, said primer will hybridize to said extended telomerase substrate and extend to form a complementary copy of said extended telomerase substrate, thereby forming duplex DNA molecules comprising an extended telomerase substrate bound to an extended primer; and
(d) correlating presence of telomerase activity in said cell sample with presence of duplex DNA molecules comprising an extended telomerase substrate bound to an extended primer and absence of telomerase activity in said cell sample with absence of said duplex DNA molecules. In some embodiments, step (c) additionally comprises steps of:
(a) heating said reaction mixture to denature said duplex DNA molecules; and
(b) cooling said reaction mixture to a temperature at which complementary nucleic acids can hybridize and said primer can extend if extended telomerase substrates are present. The heating and cooling steps may be repeated at least 5 times, and said primer is present in amounts sufficient for the formation of extended primers during each cooling step.

The method may have template-dependent DNA polymerase present in the reaction mixture of step (c) and said primer may extended by addition of nucleotides to said primer by said DNA polymerase. The template-dependent DNA polymerase is a thermostable template-dependent DNA polymerase.

The method may have template-dependent DNA ligase is present in the reaction mixture of step (c) and said primer may be extended by ligation of an oligonucleotide ligomer to said primer by said DNA ligase. The template-dependent DNA ligase may be a thermostable template-dependent DNA ligase.

In some embodiments, the telomerase substrate may be labeled. In some embodiments the primer may be labeled. In some embodiments the nucleotides added to the telomerase assay may be labeled. Where there is a label, it may be selected from the group consisting of a radioactive atom or stable isotope, a fluorescent molecule, a phosphorescent molecule, a ligand for a receptor, biotin, and avidin.

In some embodiments the telomerase substrate lacking a telomeric repeat sequence is 5′-AATCCGTCGAGCAGAGTT-3′ (SEQ ID NO:28). In some embodiments, the primer comprises a non-telomeric repeat sequence at a 5′-end of said primer. In some embodiments the primer is 5′-CCCTTACCCTTACCCTTACCCTAA-3′ (SEQ ID NO: 27), 5′-GCGCGGCTAACCCTAACCCTAACC-3′ (SEQ ID NO:32) or 5′-GCGCGGCTTACCCTTACCCTTACCCTAACC-3′ (SEQ ID NO:29).

In some embodiments, the presence of extended telomerase substrate is detected using a branched DNA probe.

In some embodiments, the method of measuring the telomerase activity further comprises normalizing the level of telomerase activity in the hair follicle cell extract relative to the amount of RNA or protein in the hair follicle cell extract. In some embodiments the amount of protein is the total amount of protein in the cell extract. In some embodiments, the amount of RNA in the cell extract is the amount of ribosomal RNA. The amount of ribosomal RNA may be determined by a PCR reaction using primers for the 18S ribosomal RNA. In other embodiments, the amount of RNA in the cell extract may be the amount of mRNA of genes which are specifically expressed in follicle cells.

The telomerase inhibitor may include an oligonucleotide having nuclease-resistant intersubunit linkages and an oligonucleotide sequence effective to bind by sequence-specific hybridization to a template region of hTR. The internucleoside linkages in the oligonucleotide may be selected from N3′→P5′ phosphoramidate and N3′→P5′ thiophosphoramidate linkages. The telomerase inhibitor may include a lipid moiety, such as a fatty acid, sterol, or derivative thereof, which is attached covalently at one end of the oligonucleotide. The oligonucleotide may be 10-20 bases in length, preferably 13-20 bases in length, and may have the sequence identified by SEQ ID NO:12 (5′-TAGGGTTAGACAA-3′). One exemplary telomerase inhibitor is the compound designated herein as GRN163L.

In a further embodiment, each exposing step (a) and (b) includes administering the telomerase inhibitor to the subject in an amount effective, when administered alone, to inhibit proliferation of cancer cells in the subject. Where the telomerase inhibitor is the compound GRN163L, it may be administered to the subject by intravenous infusion, under infusion conditions effective to produce a blood concentration of the inhibitor of between 1 nM and 100 μM.

The subject may be exposed to the telomerase inhibitor for the treatment of cancer and the cancer is selected from the group consisting of breast cancer, ovarian cancer, basal-cell carcinoma, small-cell lung carcinoma, non-small cell lung carcinoma, squamous cell carcinoma, hepatocellular carcinoma, renal cell carcinoma, and multiple myeloma.

In one embodiment the invention is directed to a method for evaluating the biological response of a subject exposed to a telomerase modulator comprising the steps of:
(a) obtaining a hair follicle from said subject;
(b) preparing a cell extract from the hair follicle by lysing the cells;
(c) incubating said cell extract in a reaction mixture comprising an exogenous telomerase substrate under conditions such that telomerase can catalyze extension of said telomerase substrate by addition of telomeric repeat sequences;
(d) replicating said extended telomerase substrate; and
(e) correlating presence of telomerase activity in said cell extract with presence of said extended telomerase substrate and absence of telomerase activity in said cell extract with absence of said extended telomerase substrate.

In another embodiment, the invention is directed to a method for evaluating the biological response of a subject exposed to a telomerase modulator comprising the steps of:
(a) obtaining a hair follicle from said subject;
(b) preparing a cell extract from the hair follicle by lysing the cells;
(c) incubating the cell extract in a reaction mixture comprising a telomerase substrate and a buffer in which telomerase can catalyze extension of said telomerase substrate by addition of telomeric repeat sequences;
(d) adding to said reaction mixture a template-dependent RNA polymerase that recognizes a promoter sequence operably linked to said telomerase substrate;
(e) allowing said RNA polymerase to form an RNA copy of said extended telomerase substrate if an extended telomerase substrate is present in said reaction mixture; and
(f) correlating presence of telomerase activity in said cell extract with presence of RNA copies of said extended telomerase substrate and absence of telomerase activity in said cell extract with absence of said RNA copies. The RNA polymerase may recognize a single-stranded nucleic acid substrate. The RNA polymerase may be selected from the group consisting of an N4 RNA polymerase, an SP6 RNA polymerase, a T7 RNA polymerase and a T3 RNA polymerase.

In another embodiment, the invention is directed to a kit for measuring telomerase activity in a subject exposed to a telomerase modulator, said kit comprising:
(a) tweezers for plucking hair;
(b) a lysis buffer;
(c) a telomerase substrate; and
(d) a primer comprising a sequence complementary to a telomeric repeat sequence. The kit may further comprises an assay buffer. The kit may further comprise an oligonucleotide control for primer extension. In the kit, the telomerase substrate may be 5′-AATCCGTCGAGCAGAGTT-3′ (SEQ ID NO:28), said primer may be 5′-GCGCGGCTTA CCCTTACCCTTACCCTAACC-3′ (SEQ ID NO:29) and said kit may further comprise 10× TRAP reaction buffer, a stock solution of dATP, dGTP, dCTP and dTTP, 5′-AATCCGTCGAGCAGAGTTAAAAGGCCGAGAAGCGAT-3′ (SEQ ID NO:30) and 5′-ATCGCTTCTCGGCCTTTT-3′ (SEQ ID NO:26).

In another embodiment, the invention is directed to a method of selecting hair follicles with live cells comprising staining the hair follicle with a DNA binding label. The method of claim 39 wherein the DNA binding label is 4′,6-diamidino-2-phenylindole (DAPI).

These and other objects and features of the invention will become more fully apparent when the following detailed description of the invention is read in conjunction with the accompanying drawings.

### BRIEF DESCRIPTION OF THE DRAWINGS

FIG. 1 is a series of photographs showing acceptable and unacceptable hair follicles for use in the assay.

FIG. 2 is a photograph of the PCR products from the TRAP Assay of human hair follicles from healthy donors showing telomerase activity.

FIG. 3A is a photograph of the acrylamide gel the telomerase products of mice who received 3 days of injections of vehicle or GRN163L. FIG. 3B is a graph of the total products generated by telomerase present in the hair follicles.

FIG. 4A is a photograph of the acrylamide gel of the telomerase products of rats who received 3 days of injections of vehicle or GRN163L. FIG. 4B is a graph of the total products generated by telomerase present in the hair follicles.

FIG. 5A is a graph of the total product generated by telomerase present in the hair follicle cells of rats who received different concentrations of GRN163L one day after dosing. FIG. 5A is a graph of the total product generated by telomerase present in the hair follicle cells of rats who received different concentrations of GRN163L four days after dosing.

FIG. 6A is a photograph of the acrylamide gel of human hair follicles which were exposed to different concentrations of GRN163L. FIG. 6B is a graph of the total products generated by telomerase present in the hair follicles.

FIG. 7 is a graph of the total products generated by telomerase present in the hair follicles of mice who received 4 weeks of injections of vehicle or GRN163L.

FIG. 8 is a graph showing the tumor volume over time in the mice that received 4 weeks of Tiw injections GRN163L.

### DETAILED DESCRIPTION OF THE INVENTION

### I. Definitions

The terms below have the following meanings unless indicated otherwise.

A “hair follicle” refers to the plucked hair of a subject which includes the hair follicle at the base of the hair, including the bulb and the sheath.

A “subject” refers to a mammal. For the purposes of this invention, the hair follicle is obtained from mammals such as humans; agriculturally important mammals, such as cattle, horses, sheep; and/or veterinary mammals, such as cats, rabbits, rodents and dogs. In rodents, cats, rabbits and dogs the hair is preferably the whisker or vibrissa hair.

A “ACX primer” is also called a “downstream primer” is composed of sequences complementary to imperfect telomeric repeats and one perfect repeat. Preferably the primer is 5′-(CCCTTA)₃CCCTAA-3′ (SEQ ID NO:30). A “primer with sequences complementary to a telomerase repeat” includes a primer that may contain one or more mismatched bases with repeat to the telomerase substrate extension product to which the primer is intended to hybridize. The number of mismatches that can be tolerated within this definition can vary depending upon the length and sequence composition of the primer, the temperature and reaction conditions employed during the PCR step.

The “telomerase substrate” or “TS” is an oligonucleotide chosen to be recognized by the mammalian telomerase to be tested in the subject. If one is using the present method to determine the level of telomerase activity in the hair follicles of a human, one employs a telomerase substrate recognized by human telomerase. Preferably when one employs a DNA polymerase based primer extension step, the telomerase substrate should not comprise a complete telomeric repeat sequence to minimize primer dimer formation. For instance, an especially preferred human telomerase substrate of the invention is oligonucleotide TS, (AATCCGTCGAGCAGAGTT) (SEQ ID NO:28) which contains a sequence at its 3′ end that is identical to five of the six bases of the human telomeric repeat but otherwise contains no complete telomeric repeat sequences. Where the replication or detection method is not compromised by the presence of repeats in the telomerase substrate, there is no requirement that the telomerase substrate be free of telomeric repeat sequence. For example, where primer extension is mediated by a ligase activity or replication is achieved by means in which specific hybridization of a probe or primer to a telomeric repeat sequence is not problematic. The telomerase substrate can be linear single stranded or duplex nucleic acids or circular plasmid DNA that undergoes linearization at a specific site, either inducibly or spontaneously.

The “DNA binding label” is a label of dye which can tightly bind to DNA. The DNA label is any label which is able to enter live hair follicle cells and bind DNA, such as fluorescent labels, UV excited labels etc. Preferably the “DNA label” is a fluorescent label such as 4′,6-diamidino-2-phenylindole (DAPI) or Hoechst stains, propidium iodide or 7-aminoactinomycinD (7-AAD). More preferably the label is DAPI.

“Telomerase activity” is the activity of a mammalian telomerase reverse transcriptase. In particular, the activity of the telomerase is the addition of telomeric DNA to a telomerase substrate per unit time.

A “telomerase modulator” is a compound that directly or indirectly either inhibits or activates the expression or activity of telomerase. A “telomerase modulator” may be a “telomerase inhibitor” or a “telomerase activator”.

A “telomerase inhibitor” is a compound that directly or indirectly inhibits or blocks the expression or activity of telomerase. A telomerase inhibitor is said to inhibit or block telomerase if the activity of the telomerase in the presence of the compound is less than that observed in the absence of the compound. Preferably the telomerase is human telomerase. More preferably, the telomerase inhibitor is an hTR template inhibitor.

An “hTR template inhibitor” is a compound that blocks the template region (the region spanning nucleotides 30-67 of SEQ ID NO: 1 herein) of the RNA component of human telomerase, thereby inhibiting the activity of the enzyme. The inhibitor is typically an oligonucleotide that is able to hybridize to this region. Preferably, the oligonucleotide includes a sequence effective to hybridize to a more specific portion of this region, having sequence 5′-CUAACCCUAAC-3′ (SEQ ID NO: 25), spanning nucleotides 46-56 of SEQ ID NO: 1 herein.

A “telomerase activator” is a compound that activates or increases the expression or activity of telomerase. A telomerase activator is said to activate or increase the activity of telomerase if the activity of the telomerase in the presence of the compound is greater than that observed in the absence of the compound. Preferably the telomerase is human telomerase. More preferably, the telomerase activator is an astragenol, cycloastragenol, protopanaxatiol or ginsenosidol as described in WO2005/000248, WO2005/044179 and WO2005/000245 all of which are incorporated herein in their entirety.

A “polynucleotide” or “oligonucleotide” refers to a ribose and/or deoxyribose nucleoside subunit polymer or oligomer having between about 2 and about 200 contiguous subunits. The nucleoside subunits can be joined by a variety of intersubunit linkages, including, but not limited to, phosphodiester, phosphotriester, methylphosphonate, P3′→N5′ phosphoramidate, N3′→P5′ phosphoramidate, N3′→P5′ thiophosphoramidate, and phosphorothioate linkages. The term also includes such polymers or oligomers having modifications, known to one skilled in the art, to the sugar (e.g., 2′ substitutions), the base (see the definition of “nucleoside” below), and the 3′ and 5′ termini. In embodiments where the oligonucleotide moiety includes a plurality of intersubunit linkages, each linkage may be formed using the same chemistry, or a mixture of linkage chemistries may be used. When an oligonucleotide is represented by a sequence of letters, such as “ATGUCCTG,” it will be understood that the nucleotides are in 5′→3′ order from left to right. Representation of the base sequence of the oligonucleotide in this manner does not imply the use of any particular type of internucleoside subunit in the oligonucleotide.

The term “nucleoside” includes the natural nucleosides, including 2′-deoxy and 2′-hydroxyl forms, e.g., as described in Komberg and Baker, DNA Replication, 2nd Ed. (Freeman, San Francisco, 1992), and analogs. “Analogs”, in reference to nucleosides, includes synthetic nucleosides having modified nucleobase moieties (see definition of “nucleobase” below) and/or modified sugar moieties, e.g., described generally by Scheit, Nucleotide Analogs (John Wiley, New York, 1980). Such analogs include synthetic nucleosides designed to enhance binding properties, e.g., stability, specificity, or the like, such as disclosed by Uhlmann and Peyman (Chemical Reviews 90:543-584, 1990). An oligonucleotide containing such nucleosides, and which typically contains synthetic nuclease-resistant internucleoside linkages, may itself be referred to as an “analog”.

A “nucleobase” includes (i) native DNA and RNA nucleobases (uracil, thymine, adenine, guanine, and cytosine), (ii) modified nucleobases or nucleobase analogs (e.g., 5-methylcytosine, 5-bromouracil, or inosine) and (iii) nucleobase analogs. A nucleobase analog is a compound whose molecular structure mimics that of a typical DNA or RNA base.

An oligonucleotide having “nuclease-resistant linkages” refers to one whose backbone has subunit linkages that are substantially resistant to nuclease cleavage, in non-hybridized or hybridized form, by common extracellular and intracellular nucleases in the body; that is, the oligonucleotide shows little or no nuclease cleavage under normal nuclease conditions in the body to which the oligonucleotide is exposed. The N3′→P5′ phosphoramidate (NP) or N3′→P5′ thiophosphoramidate (NPS) linkages described below are nuclease resistant.

The term “lipid” is used broadly herein to encompass substances that are soluble in organic solvents, but sparingly soluble, if at all, in water. The term lipid includes, but is not limited to, hydrocarbons, oils, fats (such as fatty acids and glycerides), sterols, steroids and derivative forms of these compounds. Preferred lipids are fatty acids and their derivatives, hydrocarbons and their derivatives, and sterols, such as cholesterol.

Fatty acids usually contain even numbers of carbon atoms in a straight chain (commonly 12-24 carbons) and may be saturated or unsaturated, and can contain, or be modified to contain, a variety of substituent groups. For simplicity, the term “fatty acid” also encompasses fatty acid derivatives, such as fatty or esters.

The term “hydrocarbon” encompasses compounds that consist only of hydrogen and carbon, joined by covalent bonds. The term encompasses open chain (aliphatic) hydrocarbons, including straight chain and branched hydrocarbons, and saturated as well as mono-and poly-unsaturated hydrocarbons. The term also encompasses hydrocarbons containing one or more aromatic rings.

As used herein, the term “lipid” also includes amphipathic compounds containing both lipid and hydrophilic moieties.

A “cancer” is a malignant tumor of epithelial-cell origin, that is, a malignant tumor that begins in the lining layer (epithelial cells) of organs. At least 80% of all cancers are carcinomas, and include breast cancer, both ductal and lobular carcinomas of the breast; ovarian cancer; basal-cell carcinoma, the most common non-melanoma skin cancer; squamous cell carcinoma, a common form of skin cancer and the most common type of lung cancer; hepatocellular carcinoma, the most common form of liver cancer; renal cell carcinoma, a malignant tumor located of the kidneys; and transitional cell carcinoma, a type of cancer that develops in the lining of the bladder, ureter, or renal pelvis. The cancer cells making up a carcinoma are referred to as “carcinoma cells.” Also includes in the term “cancer” are cancers of the blood cells such as leukemias, lymphomas and myelomas.

### II. Measurement of Activity

The present invention provides novel methods for the detection of telomerase activity in a subject being treated with a telomerase modulator. The methods of the present invention have been used to test for telomerase activity in mammalian hair follicles after systemic treatment of a mammal with a telomerase modulator. The methods of the present invention have also been used to test for telomerase activity in human hair follicles after local treatment of the hair follicles with a telomerase modulator. It has surprisingly been found that the level of telomerase activity in the hair follicles correlates with treatment of the subject with a telomerase modulator. A measurable reduction in telomerase activity in the hair follicle cells correlated with a reduction in tumor size in animal models. Accordingly, measurement of telomerase activity in hair follicle cells surprisingly correlates well with treatment success with telomerase modulators.

It has surprisingly been found that the level of telomerase activity in hair follicles is easily measured. The level of telomerase activity in hair follicles is relatively higher than in buccal scrapings or peripheral blood mononuclear cells.

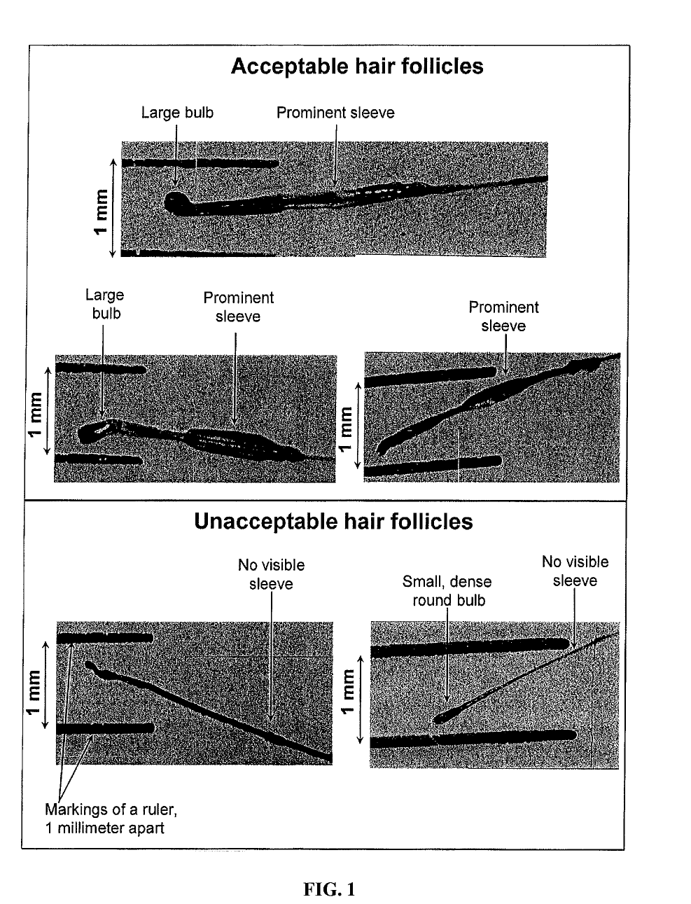

Hair follicles are obtained by plucking or pulling the hair. In rodents the vibrissa hair or whiskers are pulled. Hair can be plucked by using tweezers or other means. Furthermore, it has been found that the use of the longer whiskers or vibrissa of rodents is preferred. The hair is viewed in a microscope to identify hair with obvious follicles. Such hairs include an inner and outer root sheath region under a visible sleeve and a bulb region (See FIG. 1).

It has been found that viewing the plucked hair follicles after administration of a DNA binding label allows one to identify those plucked hairs in which the hair follicle has an abundance of live cells located in the bulge region of the hair follicle. Preferably, the DNA binding label binds to live hair follicle cells and is a fluorescent or UV excited label, for example, 4′,6′-diamindino-2-phenylimdole (DAPI). Use of only hair follicles with live cells in the sleeve region is preferred. Those hair follicles having an abundance of well stained, intact, nuclei in the sleeve region are chosen for analysis.

The hair follicle cells may be lysed using a variety of methods to release the telomerase reverse transcriptase for the assay. In particular, the cells may be lysed in a buffer comprising a detergent, selected from commercial, non-ionic and/or zwitterionic buffers. Commercial buffers include the M-PER Lysis buffer (Pierce Biotechnology, Inc., Rockford Ill.). A wide variety of non-ionic and/or zwitterionic detergents can be employed in the method. Preferred non-ionic detergents include Tween 20, Triton X-100, Triton X-114, NP-40 etc. While the exact amount of detergent is not critical, 0.1-10%, or 0.5 to 5% is typically sufficient to observe the enhanced extraction of telomerase activity.

There are no limitations on the type of assay used to measure telomerase activity. Any of the current assays for telomerase activity can be used, as well as assays that may be developed in the future. A particularly preferred method involves the analysis of telomerase activity by the Telomeric Repeat Amplification Protocol (TRAP assay).

The TRAP assay is a standard method for measuring telomerase activity in a cell extract system (Kim et al., Science 266:2011, 1997; Weinrich et al., Nature Genetics 17:498, 1997). Briefly, this assay measures the amount of nucleotides incorporated into elongation products (polynucleotides) formed by nucleotide addition to a labeled telomerase substrate or primer. This method is described in detail in U.S. Pat. Nos. 5,837,453, 5,863,726 and 5,804,380, as well as in U.S. Pat. Nos. 5,629,154 and 5,648,215, which are incorporated herein in their entirety. The use of the TRAP assay in testing the activity of telomerase inhibitory compounds is described in various publications, including WO 01/18015. In addition, the following kits are available commercially for research purposes for measuring telomerase activity: TRAPeze™ XK Telomerase Detection Kit (Intergen Co., Purchase N.Y.); and TeloTAGGG Telomerase PCR ELISA plus (Roche Diagnostics, Indianapolis Ind.).

To practice the TRAP method, one first prepares a cell extract; preferably using a detergent based extraction method and then places the cell extract or an aliquot of the cell extract in a reaction mixture comprising a telomerase substract and a buffer compatible with telomerase activity. The particular telomerase substrate chosen may vary depending on the type or origin of the telomerase activity for which one is testing. The telomerase activity expressed by one mammal may differ with respect to substrate specificity from that expressed by another mammal. Consequently, if one is using the method to determine the effect of a telomerase modulator on a human, one employs a telomerase substrate recognized by human telomerase.

When one employs a DNA polymerase-based primer extension step, the present method requires that the telomerase substrate not comprise a telomeric repeat sequence. The human telomerase adds repeats of sequence 5′-TTAGGG-3′. Thus, if one is using the present method to assay for human telomerase activity, the telomerase substrate should be a human telomerase substrate lacking the sequence 5′-TTAGGG-3′.

This requirement for the telomerase substrate to lack telomeric repeat sequences arises out of the second reaction of the present method, the non-telomerase-mediated primer extension reaction. In this reaction, an oligonucleotide primer that hybridizes only to extended telomerase substrates is added to the reaction mixture under conditions such that, if extended telomerase substrates are present, the primer binds to the extended substrates and is then extended by enzymatic action. Because telomerase can extend the telomerase substrate only by the addition of telomeric repeats, the oligonucleotide primer will necessarily comprise a sequence complementary to a telomeric repeat. If the telomerase substrate sequence employed in the telomerase extension reaction comprised a telomeric repeat, then the primer employed in the primer extension reaction could hybridize to unextended telomerase substrate, resulting in false positive results.

The primer extension reaction conducted subsequent to the telomerase substrate extension serves to amplify the signal produced by the presence of telomerase activity in a sample (extended telomerase substrates) by producing a second signal (extended primers). The primers can be extended by any means that requires the presence of extended telomerase substrates for primer extension to occur; preferred means are mediated by a template-dependent DNA or RNA polymerase, a template-dependent DNA ligase, or a combination of the two. With these means, if telomerase activity is present in the sample, an extended telomerase substrate is formed and then hybridizes to a primer, providing a substrate for either DNA or RNA polymerase or DNA ligase to produce a primer extension product.

Once a primer extension product has formed, one can disassociate (typically by heating, but one could also use an enzyme or chemical process, such as treatment with helicase) the extended primer from the extended substrate. If additional primer and primer extension reagent is present in the sample, then a new primer/extended telomerase substrate complex can form, leading to the production of another extended primer. One can repeat the process of primer extension and denaturation several to many times, depending upon the amount of signal desired. Typically, primer extension and denaturation of extended primer/extended telomerase substrate complexes will be performed at least 5, 10, 15, 20 to 30 or more times. Moreover, if a second primer complementary to the 3′-end of the extended primer is present in the reaction mixture, one can increase the signal (both extended primer and also additional extended telomerase substrate) dramatically. Unextended telomerase substrate still present in the reaction mixture during the primer extension step can function as such a second primer.

Those of skill in the art will recognize that if the primer extension reagent is a DNA polymerase, and a second primer is present, one has the requisite components for a polymerase chain reaction, more fully described in U.S. Pat. Nos. 4,683,195; 4,683,202; and 4,965,188, provided the appropriate buffer and nucleoside triphosphates are present in the reaction mixture. PCR amplification is a preferred mode for conducting the primer extension reaction step of the present invention and dramatically increases sensitivity, speed, and efficiency of detecting telomerase activity as compared to the conventional assay.

The telomerase assay is particularly well suited for providing a variety of means to quantitate the amount of telomerase in a sample. One means for obtaining quantitative information is the use of a PCR control oligonucleotide template added to each reaction mixture in a known amount. An illustrative PCR control oligonucleotide comprises, in 5′-3′ order, a telomerase substrate sequence, a spacer sequence (which can be any sequence of nucleotides or length and can alter spacing of the ladder produced by electrophoresis of reaction products produced from telomerase containing samples), a telomeric repeat sequence (typically present in multiple, i.e., 2 to 50, copies), and a sequence complementary to the primer used in the assay (and so which may simply be a portion of the telomeric repeat sequence). Of course, an oligonucleotide complementary to the control sequence defined above can also serve as the control sequence, and a double-stranded control nucleic acid can also be employed.

Alternatively, one can add a PCR control nucleic acid of any sequence to the reaction mixture in known amounts and amplify the control with primers which can be the same as or different from those used to amplify the extended telomerase substrate. The control oligonucleotide and/or the primers used to amplify the control oligonucleotide can be labelled identically to or differently from the label used to detect the telomerase extension products. Use of an internal control not only facilitates the determination of whether the assay was conducted properly but also facilitates quantitation of the telomerase activity present in the sample. The detailed protocol for conducting TRAP assays using primer and internal control is described in U.S. Pat. Nos. 5,629,154, and 5,863,726 which are incorporated herein in their entirety.

PCR normalization of the intensity of the telomerase ladder to that of the internal standard permits the assay to become linear so that accurate comparisons between samples can be made, as is described in the Examples section below. A weak signal resulting from the internal standard relative to that in other samples could indicate limiting PCR conditions, thus allowing the practitioner to choose to repeat the assay under non-limiting conditions, for example, by providing higher polymerase levels. The inclusion of the internal standard also immediately identifies potentially false negative samples.

It is also contemplated that the telomerase assay can be conducted without using an internal control oligonucleotide. It has been found that other means can be used to make accurate comparisons between samples as described below.

While PCR provides for exponential accumulation of primer extension products, even linear accumulation of primer extension products can provide useful results. Thus, one can use a single primer and merely make many copies of a single strand of the duplex nucleic acid that is produced when PCR is employed. Moreover, such copies can be made by means other than polymerase-mediated primer extension. Suitable methods include the ligase chain reaction (Barany, 1991, Proc. Natl. Acad. Sci. USA 88:189-193), nucleic acid sequence-based amplification (Compton, 1991, Nature 350: 91-92), self-sustained sequence replication (Guatelli et al., 1990, Proc. Natl. Acad. Sci. USA 87:1874-1878), strand displacement amplification (Walker et al., 1992, Proc. Natl. Acad. Sci. USA 89:392-396), and branched DNA signal amplification (Urdea, Sep. 12, 1994, Bio/Tech. 12:926-928; U.S. Pat. No. 5,124,246), although the latter method involves amplification of the signal produced upon probe hybridization to a target nucleic acid.

Moreover, a variety of different types of oligonucleotides can be used in telomerase activity assays. While the discussion above and Examples below illustrate assay methods with results obtained using oligodeoxyribonucleotide telomerase substrates and primers with DNA polymerase, the activity assay used in the present invention is not so limited. Thus, one can employ oligoribonucleotides or oligonucleotides that comprise one or more modified (i.e., synthetic or non-naturally occurring) nucleotides in the telomerase assay. In similar fashion, one can employ an RNA polymerase to extend a primer or to copy an extended telomerase substrate. These and other variations of the present method will be apparent to those of skill in the art upon consideration of this description of the invention.

Alternatively one can carry out the FlashPlate™ Assay as described in Asai et al., Cancer Research, 63:3931 3939 (2003). Briefly, the assay detects and/or measures telomerase activity by measuring the addition of TTAGGG telomeric repeats to a biotinylated telomerase substrate primer. The biotinylated products are captured on streptavidin-coated microtiter plates, and an oligonucleotide probe complementary to 3.5 telomere repeats, labeled with ³²P, is used for measuring telomerase products. Unbound probe is removed by washing, and the amount of probe annealing to the captured telomerase products is determined by scintillation counting.

Detection of the telomerase products may involve additional steps, depending on the needs of the practitioner and the particular label or detection means employed. In some instances the practitioner may first separate the reaction products from one another using gel electrophoresis. Other separation methods, i;e., chromatography or electrophoresis, can also be employed but for some purposes no separation will be performed and the detection of the extended relomerase substrates will be carried out.

The intensity of the telomerase product generated is also normalized relative to a control molecule such as, for example, RNA or total protein so that comparisons between samples can be made. This provides correction for the extraction efficiency of telomerase from the hair follicle cells allowing different samples to be compared. The activity of the telomerase is expressed as a value relative to protein amount or RNA amount. In particular, the amount of total protein or the amount of RNA can be used for normalization purposes. Where ribosomal RNA serves as the normalization control, the ribosomal RNA can be determined by a PCR reaction using primers directed to one of the ribosomal RNAs, e.g., the 18S ribosomal RNA. Alternatively the amount of mRNA in the cell extract may be determined by measuring the mRNA for genes specifically expressed in follicle cells or housekeeping genes. Alternatively the total RNA can be measured using an RNA fluorescent dye, preferably RIBOGREEN® fluorescent dye.

The level of activity measured by the telomerase assay after exposure to a telomerase modulator can be compared to the telomerase activity prior to exposure to the telomerase modulator. For example, hair follicles may be plucked from the same individual prior to exposure and the level of telomerase activity measured. Alternatively the level of activity can be compared to a standard level of activity found in the hair follicles of a number of individual subjects prior to exposure to a telomerase modulator. A difference in activity is observed when there is at least a 50% increase, at least a 2 fold increase, at least a 4 fold increase or at least a 6 fold increase in activity after exposure to a telomerase activator. A difference is activity is observed when there is less than 90% of the activity, less than 80% of the activity, less than 70% of the activity or less than 50% of the activity after exposure to a telomerase inhibitor.

III. Telomerase Inhibitors and Treatment of Cancer with a Telomerase Inhibitor

Telomerase is a ribonucleoprotein that catalyzes the addition of telomeric repeat sequences (having the sequence 5′-TTAGGG-3′ in humans) to chromosome ends. A variety of cancer cells have been shown to be telomerase-positive, including cells from cancer of the skin, connective tissue, adipose, breast, lung, stomach, pancreas, ovary, cervix, uterus, kidney, bladder, colon, prostate, central nervous system (CNS), retina and hematologic tumors (such as myeloma, leukemia and lymphoma). Targeting of telomerase can be effective in providing treatments that discriminate between malignant and normal cells to a high degree, avoiding many of the deleterious side effects that can accompany chemotherapeutic regimens which target dividing cells indiscriminately.

Inhibitors of telomerase identified to date include oligonucleotides, preferably oligonucleotides having nuclease resistant linkages, as well as small molecule compounds.

### A. Small Molecule Compounds

Small molecule inhibitors of telomerase include, for example, BRACO19 ((9-(4-(N,N-dimethylamino)phenylamino)-3,6-bis(3-pyrrolodino propionamido)acridine (see Mol. Pharmacol. 61(5):1154-62, 2002); DODC (diethyloxadicarbocyanine), and telomestatin. These compounds may act as G-quad stabilizers, which promote the formation of an inactive G-quad configuration in the RNA component of telomerase. Other small molecule inhibitors of telomerase include BIBR1532 (2-[(E)-3-naphthen-2-yl but-2-enoylamino]benzoic acid) (see Ward & Autexier, Mol. Pharmacol. 68:779-786, 2005; also J. Biol. Chem. 277(18):15566-72, 2002); AZT and other nucleoside analogs, such as ddG and ara-G (see, for example, U.S. Pat. Nos. 5,695,932 and 6,368,789), and certain thiopyridine, benzo[b]thiophene, and pyrido[b]thiophene derivatives, described by Gaeta et al. in U.S. Pat. Nos. 5,767,278, 5,770,613, 5,863,936, 5,656,638 and 5,760,062. One example is 3-chlorobenzo[b]thiophene-2-carboxy-2′-[(2,5-dichlorophenyl amino)thia]hydrazine, described in U.S. Pat. No. 5,760,062.

### B. Oligonucleotide-Based Telomerase Inhibitors: Sequence and Composition

The genes encoding both the protein and RNA components of human telomerase have been cloned and sequenced (see U.S. Pat. Nos. 6,261,836 and 5,583,016, respectively, both of which are incorporated herein by reference). Oligonucleotides can be targeted against the mRNA encoding the telomerase protein component (the human form of which is known as human telomerase reverse transcriptase, or hTERT) or the RNA component of the telomerase holoenzyme (the human form of which is known as human telomerase RNA, or hTR).

The nucleotide sequence of the RNA component of human telomerase (hTR) is shown in the Sequence Listing below (SEQ ID NO: 1), in the 5′→3′ direction. The sequence is shown using the standard abbreviations for ribonucleotides; those of skill in the art will recognize that the sequence also represents the sequence of the cDNA, in which the ribonucleotides are replaced by deoxyribonucleotides, with uridine (U) being replaced by thymidine (T). The template sequence of the RNA component is located within the region defined by nucleotides 46-56 (5′-CUAACCCUAAC-3′), which is complementary to a telomeric sequence composed of about one-and-two-thirds telomeric repeat units. The template region functions to specify the sequence of the telomeric repeats that telomerase adds to the chromosome ends and is essential to the activity of the telomerase enzyme (see e.g. Chen et al., Cell 100: 503-514, 2000; Kim et al., Proc. Natl. Acad. Sci. USA 98 (14):7982-7987, 2001). The design of antisense, ribozyme or small interfering RNA (siRNA) agents to inhibit or cause the destruction of mRNAs is well known (see, for example, Lebedeva, I, et al. Annual Review of Pharmacology and Toxicology, Vol. 41: 403-419, April 2001; Macejak, D, et al., Journal of Virology, Vol. 73 (9): 7745-7751, September 1999, and Zeng, Y. et al., PNAS Vol. 100 (17) p. 9779-9784, Aug. 19, 2003) and such agents may be designed to target the hTERT mRNA and thereby inhibit production of hTERT protein in a target cell, such as a cancer cell (see, for example, U.S. Pat. Nos. 6,444,650 and 6,331,399).

Oligonucleotides targeting hTR (that is, the RNA component of the enzyme) act as inhibitors of telomerase enzyme activity by blocking or otherwise interfering with the interaction of hTR with the hTERT protein, which interaction is necessary for telomerase function. See, for example, Villeponteau et al., U.S. Pat. No. 6,548,298.

A preferred target region of hTR is the template region, spanning nucleotides 30-67 of SEQ ID NO:1. Oligonucleotides targeting this region are referred to herein as “hTR template inhibitors” (see e.g. Herbert et al., Oncogene 21(4):638-42 (2002).) Preferably, such an oligonucleotide includes a sequence which is complementary or near-complementary to some portion of the 11-nucleotide region having sequence 5′-CUAACCCUAAC-3′, spanning nucleotides 46-56 of SEQ ID NO: 1.

Another preferred target region is the region spanning nucleotides 137-179 of hTR (see Pruzan et al., Nucl. Acids Research, 30:559-568, 2002). Within this region, the sequence spanning 141-153 is a preferred target. PCT publication WO 98/28442 describes the use of oligonucleotides of at least 7 nucleotides in length to inhibit telomerase, where the oligonucleotides are designed to be complementary to accessible portions of the hTR sequence outside of the template region, including nucleotides 137-196, 290-319, and 350-380 of hTR. Preferred hTR targeting sequence are given below, and identified by SEQ ID NOS: 2-20.

The region of the therapeutic oligonucleotide that is targeted to the hTR sequence is preferably exactly complementary to the corresponding hTR sequence. While mismatches may be tolerated in certain instances, they are expected to decrease the specificity and activity of the resultant oligonucleotide conjugate. In particular embodiments, the base sequence of the oligonucleotide is thus selected to include a sequence of at least 5 nucleotides exactly complementary to the hTR target, and enhanced telomerase inhibition may be obtained if increasing lengths of complementary sequence are employed, such as at least 8, at least 10, at least 12, at least 13 or at least 15 nucleotides exactly complementary to the hTR target. In other embodiments, the sequence of the oligonucleotide includes a sequence of from at least 5 to 20, from at least 8 to 20, from at least 10 to 20 or from at least 10 to 15 nucleotides exactly complementary to the hTR target sequence.

Optimal telomerase inhibitory activity may be obtained when the full length of the oligonucleotide is selected to be complementary to the hTR target sequence. However, it is not necessary that the full length of the oligonucleotide is exactly complementary to the target sequence, and the oligonucleotide sequence may include regions that are not complementary to the target sequence. Such regions may be added, for example, to confer other properties on the compound, such as sequences that facilitate purification. Alternatively, an oligonucleotide may include multiple repeats of a sequence complementary to an hTR target sequence.

The method includes administering to the subject an oligonucleotide telomerase inhibitor of the type composed of an oligonucleotide having nuclease-resistant intersubunit linkages and an oligonucleotide sequence effective to bind by sequence-specific hybridization to a template region of hTR. Preferably, the amount of the telomerase inhibitor is effective to inhibit the proliferation of cancer cells in the subject when the telomerase inhibitor is administered alone.

The oligonucleotide may be 10-20 bases in length. Preferably, the oligonucleotide is 13-20 bases in length and includes the sequence identified by SEQ ID NO: 12 (5′-TAGGGTTAGACAA-3′). An exemplary telomerase inhibitor is the compound identified as GRN163L, or an analog thereof. This compound has (i) N3′→P5′ thiophosphoramidate internucleoside linkages; (ii) the sequence identified as SEQ ID NO:12; and (iii) a palmitoyl (C16) moiety linked to the 5′ end of the oligonucleotide through a glycerol or aminoglycerol linker.

If the oligonucleotide is to include regions that are not complementary to the target sequence, such regions are typically positioned at one or both of the 5′ or 3′ termini. Exemplary sequences targeting human telomerase RNA (hTR) include the following:

| | Region of | SEQ |
|---|---|---|
| hTR Targeting Sequence | SEQ ID NO:1 | ID NO: |
| ACATTTTTTGTTTGCTCTAG | 160-179 | 2 |
| GCTCTAGAATGAACGGTGGAAGGCGGCAGG | 137-166 | 3 |
| GTGGAGGCGGCAGG | 137-151 | 4 |
| GGAAGGCGGCAGG | 137-149 | 5 |
| GTGGAAGGCGGCA | 139-151 | 6 |
| GTGGAAGGCGG | 141-151 | 7 |
| CGGTGGAAGGCGG | 141-153 | 8 |
| ACGGTGGAAGGCG | 142-154 | 9 |
| AACGGTGGAAGGCGGC | 143-155 | 10 |
| ATGAACGGTGGAAGGCGG | 144-158 | 11 |
| TAGGGTTAGACAA | 42-54 | 12 |
| CAGTTAGGGTTAG | 46-58 | 13 |
| TAGGGTTAGACA | 42-53 | 14 |
| TAGGGTTAGAC | 42-52 | 15 |
| GTTAGGGTTAG | 46-56 | 16 |
| GTTAGGGTTAGAC | 44-56 | 17 |
| GTTAGGGTTAGACAA | 42-56 | 18 |
| GGGTTAGAC | 44-52 | |
| CAGTTAGGG | 50-58 | |
| CCCTTCTCAGTT | 54-65 | 19 |
| CGCCCTTCTCAG | 56-67 | 20 |

The internucleoside linkages in the oligonucleotide may include any of the available oligonucleotide chemistries, e.g. phosphodiester, phosphotriester, methylphosphonate, P3′→N5′ phosphoramidate, N3′→P5′ phosphoramidate, N3′→P5′ thiophosphoramidate, and phosphorothioate. Typically, but not necessarily, all of the internucleoside linkages within the oligonucleotide will be of the same type, although the oligonucleotide component may be synthesized using a mixture of different linkages.

In preferred embodiments, the oligonucleotide has at least one N3′→P5′ phosphoramidate (NP) or N3′→P5′ thiophosphoramidate (NPS) linkage, which linkage may be represented by the structure: 3′-(-NH—P(═O)(—XR)—O-)-5′, wherein X is O or S and R is selected from the group consisting of hydrogen, alkyl, and aryl; and pharmaceutically acceptable salts thereof, when XR is OH or SH. More preferably, the oligonucleotide includes all NP or, most preferably, all NPS linkages.

A particularly preferred sequence for an hTR template inhibitor oligonucleotide is the sequence complementary to nucleotides 42-54 of SEQ ID NO: 1 above. The oligonucleotide having this sequence (TAGGGTTAGACAA) SEQ ID NO:12 and N3′→P5′ thiophosphoramidate (NPS) linkages is designated herein as GRN163. See, for example, Asai et al, Cancer Research 63:3931-3939 (2003); Gryaznov et al., Nucleosides Nucleotides Nucleic Acids 22(5-8):577-81 (2003).

The oligonucleotide GRN163 administered alone has shown inhibitory activity in vitro in cell culture, including epidermoid carcinoma, breast epithelium, renal carcinoma, renal adenocarcinoma, pancreatic, brain, colon, prostate, leukemia, lymphoma, myeloma, epidermal, cervical, ovarian and liver cancer cells.

The oligonucleotide GRN163 has also been tested and shown to be therapeutically effective in a variety of animal tumor models, including ovarian and lung, both small cell and non-small cell.

### C. Lipid-Oligonucleotide Conjugates

Preferably, the oligonucleotide-based enzyme inhibitor includes at least one covalently linked lipid group (see US Publication. No. 2005/0113325, which is incorporated herein by reference). This modification provides superior cellular uptake properties, such that an equivalent biological effect may be obtained using smaller amounts of the conjugated oligonucleotide compared to the unmodified form. When applied to the human therapeutic setting, this may translate to reduced toxicity risks, and cost savings.

The lipid group L is typically an aliphatic hydrocarbon or fatty acid, including derivatives of hydrocarbons and fatty acids, with examples being saturated straight chain compounds having 14-20 carbons, such as myristic (tetradecanoic) acid, palmitic (hexadecanoic) acid, and stearic (octadeacanoic) acid, and their corresponding aliphatic hydrocarbon forms, tetradecane, hexadecane and octadecane. Examples of other suitable lipid groups that may be employed are sterols, such as cholesterol, and substituted fatty acids and hydrocarbons, particularly polyfluorinated forms of these groups. The scope of the lipid group L includes derivatives such as amine, amide, ester and carbamate derivatives. The type of derivative is often determined by the mode of linkage to the oligonucleotide, as exemplified below.

In one exemplary structure, the lipid moiety is palmitoyl amide (derived from palmitic acid), conjugated through an aminoglycerol linker to the 5′ thiophosphate group of an NPS-linked oligonucleotide. The NPS oligonucleotide having the sequence shown for GRN163 and conjugated in this manner (as shown below) is designated GRN163L herein. In a second exemplary structure, the lipid, as a palmitoyl amide, is conjugated through the terminal 3′ amino group of an NPS oligonucleotide.

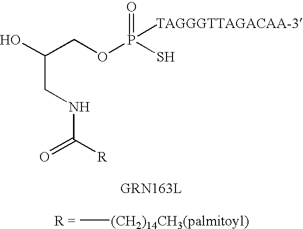

As shown in Table 1, conjugation of a single fatty acid-type lipid significantly increased telomerase inhibitory activity in cell systems relative to the unconjugated oligonucleotide.

TABLE 1
Inhibition of Telomerase by Lipid-Conjugated NPS Oligonucleotides
(based on GRN163)

| | Tm (° C.) of duplex | IC₅₀ in vitro, |
|---|---|---|
| Lipid Substitution | with RNA | HT-3 cells, nM |
| none (GRN163) | 70.0 | 1600 |
| 3′-palmitic (GRN163L) | 66.5 | 160 |
| 3′-stearic | 67.1 | 140 |
| 3′-(bis)stearic | ~40 | 1960 |
| 3′-oleic | 66.8 | 930 |
| NH-C₁₆ (palmitoyl) on 3^{rd} 5′ | 62.6 | 500 |
| residue (G) | | |
| 5′-palmitic | 65.5 | 112 |
| 3′-palmitic-5′-palmitic | 61.3 | ~10000 |
| 3′-trityl | 66.1 | 3000 |

The conjugated oligonucleotide GRN163L had significantly greater telomerase inhibiting activity in vivo than the unconjugated GRN163 following i.v. administration.

Administration of GRN163L inhibited tumor growth in mice (A549-luc IV lung metastases model) for at least 4 weeks after i.v. injection of cancer cells. The dosage was 1 μM biweekly for 5 weeks prior to injection of cancer cells, followed by 5 mg/kg twice weekly after injection. Controls showed substantial tumor growth, but none was apparent in the GRN163L-treated mouse.

### IV. Administration

The carcinoma should also be one that is responsive to cancer-cell inhibition by telomerase inhibition. As noted above, oligonucleotide telomerase inhibitors, as exemplified by GRN163 and GRN163 L, have shown inhibitory activity in vitro against human kidney, lung, pancreatic, brain, colon, prostate, breast, leukemia, lymphoma, myeloma, epidermal, cervical, ovarian and liver cancer cells, and in vivo, via local and systemic delivery, against human brain, prostate, lymphoma, myeloma, cervical, lung, and liver cancer cells. Other preferred targets include small cell lung, esophageal, head and neck, and stomach cancers.

The dose administered and the dosing schedule will follow, for example, known or recommended doses for the inhibitor employed, as indicated, for example, in the drug product insert or published clinical or animal-model data.

The therapeutic protocol for administering the telomerase inhibitor in the therapy will depend on various factors including, but not limited to, the type of cancer, the age and general health of the patient, the aggressiveness of disease progression, the TRF length (terminal restriction fragment length) and telomerase activity of the diseased cells to be treated, and the ability of the patient to tolerate the agents that comprise the combination.

In general, treatment of all carcinoma and hematological malignancy types is contemplated. In selected embodiments, the target disease comprises a solid tumor; in other embodiments, the target disease comprises a hematological malignancy. An exemplary course of treatment involves multiple doses. Sequence of combination treatments will be determined by clinical compliance criteria and/or preclinical or clinical data supporting dose optimization strategies to augment efficacy or reduce toxicity of the combination treatment. The time between dosages may be for a period from about 1-6 hours, to about 6-12 hours, to about 12-24 hours, to about 1-2 days, to about 1-2 wk or longer following the initiation of treatment. During a course of treatment, the need to complete the planned dosings may be re-evaluated.

The compounds may be administered by direct injection of a tumor or its vasculature. Alternatively, the tumor may be infused or perfused with the therapeutic compounds using any suitable delivery vehicle. The compounds may be administered locally to an affected organ. Systemic administration may also be performed. Continuous administration may be applied where appropriate; for example, where a tumor is excised and the tumor bed is treated to eliminate residual disease. Delivery via syringe or catheterization is preferred. Such continuous perfusion may take place for a period from about 1-6 hours, to about 6-12 hours, to about 12-24 hours, to about 1-2 days, to about 1-2 weeks or longer following the initiation of treatment. Generally, the dose of the therapeutic composition via continuous perfusion will be equivalent to that given by a single or multiple injections, adjusted over a period of time during which the perfusion occurs.

The therapeutic agents are administered to a subject, such as a human patient, in a formulation and in an amount effective to achieve a clinically desirable result. For the treatment of cancer, desirable results include reduction in tumor mass (as determined by palpation or imaging; e.g., by radiography, radionucleotide scan, CAT scan, or MRI), reduction in the rate of tumor growth, reduction in the rate of metastasis formation (as determined e.g., by histochemical analysis of biopsy specimens), reduction in biochemical markers (including general markers such as ESR, and tumor specific markers such as serum PSA), and improvement in quality of life (as determined by clinical assessment, e.g., Karnofsky score), increased time to progression, disease-free survival and overall survival.

The amount of each agent per dose and the number of doses required to achieve such effects will vary depending on many factors including the disease indication, characteristics of the patient being treated and the mode of administration. Typically, the formulation and route of administration will provide a local concentration at the disease site of between 1 nM and 100 μM of each agent. The physician will be able to vary the amount of the compounds, the carrier, the dosing frequency, and the like, taking into consideration such factors as the particular neoplastic disease state and its severity; the overall condition of the patient; the patient's age, sex, and weight; the mode of administration; the suitability of concurrently administering systemic anti-toxicity agents; monitoring of the patient's vital organ functions; and other factors typically monitored during cancer chemotherapy. In general, the compounds are administered at a concentration that affords effective results without causing excessive harmful or deleterious side effects.

### V. Formulations

The pharmaceutical carrier(s) employed may be solid or liquid. Liquid carriers can be used in the preparation of solutions, emulsions, suspensions and pressurized compositions. The compounds are dissolved or suspended in a pharmaceutically acceptable liquid excipient. Suitable examples of liquid carriers for parenteral administration include water (which may contain additives, e.g., cellulose derivatives, preferably sodium carboxymethyl cellulose solution), phosphate buffered saline solution (PBS), alcohols (including monohydric alcohols and polyhydric alcohols, e.g., glycols) and their derivatives, and oils (e.g., fractionated coconut oil and arachis oil). The liquid carrier can contain other suitable pharmaceutical additives including, but not limited to, the following: solubilizers, suspending agents, emulsifiers, buffers, thickening agents, colors, viscosity regulators, preservatives, stabilizers and osmolarity regulators.

For parenteral administration, the carrier can also be an oily ester such as ethyl oleate and isopropyl myristate. Sterile carriers are useful in sterile liquid form compositions for parenteral administration. Sterile liquid pharmaceutical compositions, solutions or suspensions can be utilized by, for example, intraperitoneal injection, subcutaneous injection, intravenously, or topically. The compositions can also be administered intravascularly or via a vascular stent.

The liquid carrier for pressurized compositions can be a halogenated hydrocarbon or other pharmaceutically acceptable propellant. Such pressurized compositions may also be lipid encapsulated for delivery via inhalation. For administration by intranasal or intrabronchial inhalation or insufflation, the compositions may be formulated into an aqueous or partially aqueous solution, which can then be utilized in the form of an aerosol.

The compositions may be administered topically as a solution, cream, or lotion, by formulation with pharmaceutically acceptable vehicles containing the active compound. The compositions of this invention may be orally administered in any acceptable dosage including, but not limited to, formulations in capsules, tablets, powders or granules, and as suspensions or solutions in water or non-aqueous media. Pharmaceutical compositions and/or formulations comprising the oligonucleotides of the present invention may include carriers, lubricants, diluents, thickeners, flavoring agents, emulsifiers, dispersing aids or binders. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Modes of administration and formulation may be dependent on the drug and its approved mode of administration. When the telomerase inhibitor is GRN163L, formulation in 0.9% sodium chloride (normal saline) and administration by i.v. is a preferred route, preferably via infusion over 2-8 hours, e.g. a 6 hr infusion. While the lipid-conjugated oligonucleotides described herein, such as GRN163L, have superior characteristics for cellular and tissue penetration, these and other compounds may be formulated to provide further benefit in this area, e.g. in liposome carriers. The use of liposomes to facilitate cellular uptake is described, for example, in U.S. Pat. Nos. 4,897,355 and 4,394,448, and numerous publications describe the formulation and preparation of liposomes. Liposomal formulations can also be engineered, by attachment of targeting ligands to the liposomal surface, to target sites of neovascularization, such as tumor angiogenic regions. The compounds can also be formulated with additional penetration/transport enhancers, such as unconjugated forms of the lipid moieties described above, including fatty acids and their derivatives. Examples include oleic acid, lauric acid, capric acid, myristic acid, palmitic acid, stearic acid, linoleic acid, linolenic acid, dicaprate, tricaprate, recinleate, monoolein (a.k.a. 1-monooleoyl-rac-glycerol), dilaurin, caprylic acid, arichidonic acid, glyceryl 1-monocaprate, 1-dodecylazacycloheptan-2-one, acylcamitines, acylcholines, mono- and di-glycerides and physiologically acceptable salts thereof (i.e., oleate, laurate, caprate, myristate, palmitate, stearate, linoleate, etc.). Other useful adjuvants include substrates for transendothelial migration, such as glucose uptake systems for facilitated egress from the vascular space to the tumor microenvironment.

VI Telomerase Activators and Treatment of Various Diseases with a Telomerase Activator

Various compounds have been described as telomerase activators. For example, the compounds described in International Patent Publication Nos. WO2005/000245, WO2005/000248 and WO2005/044179 which are incorporated herein in their entirety.

Certain plant species, e.g. Astragalus or Cimicifuga species produce compounds which activate telomerase in cells. In particular, a extract of Astragalus membranaceus may be obtained by the method set forth in WO2005/044179. The concentration of the extract in the formulation is preferably in the range of 0.1 to 100% (w/v). The extract may also be provided directly in solid form, without a vehicle or excipients. The extract may be formulated in a vehicle that is appropriate for the relevant application, whether it be a cosmetic for topical use, nutraceutical for ingestion or pharmaceutical for administration by a variety of routes including topical, oral or injection.

Other compounds include those described in International Patent Publication Nos. WO2005/000245 and WO2005/000248. Specifically, the compounds of the formula In compounds of formula I:

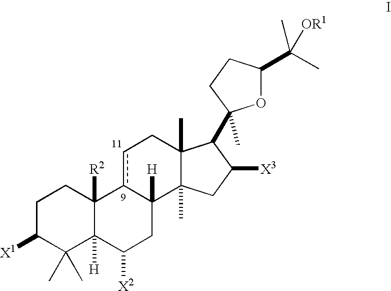

each of X¹, X², and X³ is independently selected from hydroxy, lower alkoxy, lower acyloxy, keto, and a glycoside;

OR¹ is selected from hydroxy, lower alkoxy, lower acyloxy, and a glycoside;

wherein any of the hydroxyl groups on said glycoside may be substituted with a further glycoside, lower alkyl, or lower acyl, such that the compound includes a maximum of three glycosides; and

R² is methyl and [symbol] represents a double bond between carbons 9 and 11; or, in preferred embodiments, R² forms, together with carbon 9, a fused cyclopropyl ring, and [symbol] represents a single bond between carbons 9 and 11.

Preferably, the compound includes zero, one, or two, more preferably zero or two, glycosides, none of which is substituted with a further glycoside. Preferably, glycosides are of the D (naturally occurring) configuration.

In selected embodiments of formula I, each of X¹ and X² is independently selected from hydroxy, lower alkoxy, lower acyloxy, and a glycoside, and X³ is selected from hydroxy, lower alkoxy, lower acyloxy, keto, and a glycoside. In further embodiments, X¹ is OH or a glycoside, each of X² and OR¹ is independently OH or a glycoside, and X³ is OH or keto. Exemplary compounds of formula I include astragaloside IV, cycloastragenol, astragenol, astragaloside IV 16-one, cycloastragenol 6-β-D-glucopyranoside, and cycloastragenol 3-β-D-xylopyranoside. In selected embodiments, the compound is selected from astragaloside IV, cycloastragenol, astragenol, and astragaloside IV 16-one. In one embodiment, the compound is astragaloside IV.

In compounds of formula II:

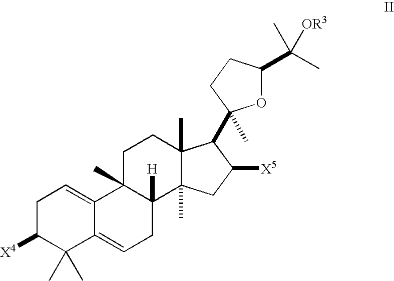

each of X⁴ and X⁵ is independently selected from hydroxy, lower alkoxy, lower acyloxy, keto, and a glycoside, and

OR³ is selected from hydroxy, lower alkoxy, lower acyloxy, and a glycoside, wherein any of the hydroxyl groups on said glycoside may be substituted with a further glycoside, lower alkyl, or lower acyl, such that the compound includes a maximum of three glycosides.

Preferably, the compound includes zero, one, or two glycosides, none of which is substituted with a further glycoside; glycosides are preferably of the D configuration.

In selected embodiments of formula II, each of X⁴ and OR³ is selected from hydroxy, lower alkoxy, lower acyloxy, and a glycoside, and X⁵ is selected from hydroxy, lower alkoxy, lower acyloxy, and keto (═O). In further embodiments, X⁴ is OH or a glycoside, and each of X⁵ and OR³ is OH. In one embodiment, X⁴ is OH.

In compounds of formula III:

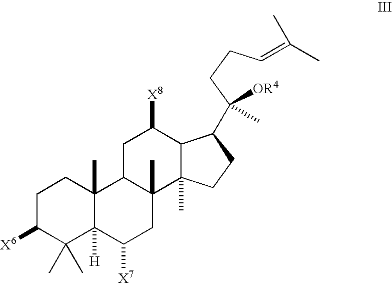

each of X⁶, X⁷, and X⁸ is independently selected from hydroxy, lower alkoxy, lower acyloxy, keto, and a glycoside, and

OR⁴ is selected from hydroxy, lower alkoxy, lower acyloxy, and a glycoside,

wherein any of the hydroxyl groups on said glycoside may be substituted with a further glycoside, lower alkyl, or lower acyl, such that the compound includes a maximum of three glycosides.

Preferably, the compound includes zero, one, or two glycosides, none of which is substituted with a further glycoside; glycosides are preferably of the D configuration.

In selected embodiments of formula III, each of X⁶, X⁷, X⁸ and OR⁴ is independently selected from hydroxy, lower alkoxy, lower acyloxy, and a glycoside, and is preferably selected from hydroxy and a glycoside. In further embodiments, each of X⁸ and OR⁴ is OH, and each of X⁶ and X⁷ is independently selected from hydroxyl and a glycoside. In still further embodiments, each of OR⁴, X⁶ and X⁸ is OH, and X⁷ is a glycoside. An exemplary compound of formula III is ginsenoside RH1.

The range of beneficial effects that may be achieved by telomerase activation include, for example: more rapid wound healing, the slowing of telomere loss occurring during aging of cells; postponing or reversing cellular senescence in disease conditions associated with cellular senescence; treating a disease condition associated with cells having a higher rate of cell division than normal cells of that cell type; treating a disease condition in which one or more cell types are limiting and reducing telomere repeat loss while expanding cell number. Disease conditions subject to treatment by an increase in telomerase activity include HIV infection and degenerative disease, such as neurodegenerative disease, degenerative disease of the bones or joints, macular degeneration, atherosclerosis and anemia, as well as wounds or other acute or chronic conditions of the epidermis.

The following examples are offered by way of illustration and not by way of limitation.

### EXAMPLES

### Example 1

### Preparation and Lipid Conjugation of Oligonucleotide N3′→-P5′ Phosphoramidates or N3′→-P5′ Thiophosphoramidates

These compounds may be prepared as described, for example, in McCurdy et al., Tetrahedron Letters 38:207-210 (1997) or Pongracz & Gryaznov, Tetrahedron Letters 49:7661-7664 (1999). The starting 3′-amino nucleoside monomers may be prepared as described in Nelson et al., J. Org. Chem. 62:7278-7287 (1997) or by the methods described in Gryaznov et al., US Appn. Pubn. No. 2006/0009636.

A variety of synthetic approaches can be used to conjugate a lipid moiety L to the oligonucleotide, depending on the nature of the linkage selected; see, for example, Mishra et al., Biochim. et Biophys. Acta 1264:229-237 (1995), Shea et al., Nucleic Acids Res. 18:3777-3783 (1995), or Rump et al., Bioconj. Chem. 9:341-349 (1995). Typically, conjugation is achieved through the use of a suitable functional groups at an oligonucleotide terminus. For example, the 3′-amino group present at the 3′-terminus of the NP and NPS oligonucleotides can be reacted with carboxylic acids, acid chlorides, anhydrides and active esters, using suitable coupling catalysts, to form an amide linkage. Thiol groups are also suitable as functional groups (see Kupihar et al., Bioorg. Med. Chem. 9:1241-1247 (2001)). Various amino- and thiol-functionalized modifiers of different chain lengths are commercially available for oligonucleotide synthesis.

Specific approaches for attaching lipid groups to a terminus of an NP or NPS oligonucleotide include those described in US Appn. Pubn. No. 2005/0113325, which is incorporated herein by reference. In addition to the amide linkages noted above, for example, lipids may also be attached to the oligonucleotide chain using a phosphoramidite derivative of the lipid, to produce a phosphoramidate or thiophosphoramidate linkage connecting the lipid and the oligonucleotide. The free 3′-amino group of the fully protected support-bound oligonucleotide may also be reacted with a suitable lipid aldehyde, followed by reduction with sodium cyanoborohydride, which produces an amine linkage.

For attachment of a lipid to the 5′ terminus, as also described in US Appn. Pubn. No. 2005/0113325, the oligonucleotide can be synthesized using a modified, lipid-containing solid support. Reaction of 3-amino-1,2-propanediol with a fatty acyl chloride (RC(O)Cl), followed by dimethoxytritylation of the primary alcohol and succinylation of the secondary alcohol, provides an intermediate which is then coupled, via the free succinyl carboxyl group, to the solid support. An example of a modified support is shown below, where S— represents a long chain alkyl amine CPG support, and R represents a lipid.

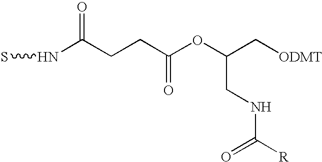

This procedure is followed by synthesis of the oligonucleotide in the 5′ to 3′ direction, as described, for example, in Pongracz & Gryaznov (1999), starting with deprotection and phosphitylation of the —ODMT group. This is effective to produce, for example, the following structure, after cleavage from the solid support:

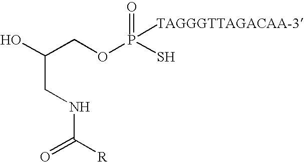

The structure above, when —R is —(CH₂)₁₄CH₃ (palmitoyl), is designated herein as GRN163L.

### Example 2

### Human Hair Follicle Collection Procedure

Hair samples were collected from the scalp of healthy donor humans. Using a stainless steel Slant Tweezer (Tweezerman Professional, model 208123) 5-7 hairs were tightly grasped as close to the scalp as possible. The hairs were plucked out of the scalp with a quick motion in the direction of the emergence of the hair from the scalp.

The hairs were carefully separated using pointed forceps. The individual hair follicles at the end of each hair were inspected by eye. If a hair follicle was visible by eye, it was used. If the hair follicle was not visible or if the hair shaft was broken, the hair sample was discarded. FIG. 1 shows examples of acceptable and not acceptable hair follicles. Acceptable hair follicles have a large bulb and a prominent intact sleeve.

For those hairs having intact hair follicles, the hair shaft was trimmed with scissors, leaving approximately 1 cm of the hair shaft above the hair follicle with the intact follicle at one end. The trimmed hair follicle sample was placed follicle end first into a test tube containing 1 mL Freezing Medium (90% fetal bovine serum, 10% DMSO (American Type Culture Collection, Virginia) The fetal bovine serum (Hyclone, Utah) used in the Freezing Medium had previously been heat inactivated at 56° C. for 30 minutes. The hair sample was completely submerged in the Freezing Medium and the vial tightly closed. The vial was placed on dry ice.

### Example 3

### Staining Hair Follicles with 4′,6-diamidino-2-phenylindole (DAPI) for Inspection by Fluorescent Microscopy

The single hair follicles obtained from Example 2 were lysed in M-PER Lysis Buffer (Pierce Biotechnology, Illinois) by immersing the follicle in the buffer and incubating the tube on ice for 5-6 hours with intermittent flicking.

The hair was retrieved from the lysis buffer using forceps, picking up the hair by the shaft and not by the bulb. The hair was placed on a slide and approximately 20 μL of DAPI solution (1 μg/mL) was placed over the follicle, taking care to cover the bulb. DAPI Solution is prepared from DAPI Stock Solution (1 mg/mL) [Molecular Probes, Oregon] at a 1:1000 dilution to 1 μg/mL in Phosphate Buffered Saline (PBS). The DAPI Stock Solution is stored at −20° C. in the dark. The slide was protected from direct light. A micro cover glass was placed over the hair follicle taking care not to trap air bubbles. The edges of the cover glass were sealed with nail polish.

The hair follicle was inspected under epifluorescence microscope after being stained for a few minutes. The epifluorescent microscope filter being set for DAPI excitation/emission.

Although DAPI is generally considered to only stain non-viable and fixed cells, it has surprisingly been found that the DAPI enters the live hair follicle cells. Hair follicles which have abundant cells in the bulge region (under the sleeve) of the hair follicle are chosen for analysis. It has surprisingly been found that by choosing hair follicles which have abundant live cells in the bulge region more accurate measurements of the telomerase activity in the hair follicle cells can be determined.

On the other hand, if there are only cells present in the bulb region of the hair follicle, the hair follicle is not analyzed.

### Example 4

### Two-Step TRAP Assay in Hair Follicles from Healthy Human Donors

The single hair follicles obtained from Example 2 were lysed in 30 μL of M-PER Lysis Buffer™ (Pierce Biotechnology, Illinois) by immersing the follicle in the buffer and incubating the tube on ice for 5-6 hours with intermittent flicking.

PCR Mix-1 was prepared from 5 μL of 10× TRAP Buffer with BSA (200 mM Tris-HCl pH 8.3, 15 mM MgCl₂, 630 mM KCl, 10 mM EGTA, 1 mg/mL BSA, and 0.5% Tween-20); 1.0 μL of 2.5mM dNTP; 0.1 μL of 0.5 mg/mL (85 μM) Cy5-TS sequence (Cy5-AATCCGTCGAGCAGAGTT) (SEQ ID NO:28); 1.0 μL of 0.1 mg/mL (18 μM) U2 primer (ATCGCTTCTCGGCCTTTT) (SEQ ID NO:26); 31.0 μL milliQ water; 5.0 μL M-PER lysis buffer (Pierce Biotechnology, Illinois)

5 μL of the hair follicle lysate was placed into the PCR plate (Stratgene, Calif.). 43.1 μL of PCR Mix-1 was added to each well. The PCR plate was sealed with adhesive film and the telomerase extension reaction was run for 30 minutes at 30° C.

After the telomere extension was completed, the temperature of the PCR machine was raised to 40° C. and 1.9 μL of PCR Mix-2 was quickly added to each well. PCR Mix-2 was prepared from 1.0 μL of 0.1 mg/mL (11 μM) ACX primer (GCGCGGCTTACCCTTACCCTTACCCTAACC) (SEQ ID NO:29); 0.5 μL of 0.1 fmol/mL TSU2 internal control template (AATCCGTCGAGCAGAGTTAAAAGGCCGAGAAGCGAT) (SEQ ID NO:30); and 0.4 μL of 5 U/μL AmpliTaq polymerase (Applied Biosystems, California). The PCR plate was resealed and the PCR machine was allowed to cycle (30 seconds at 94° C., 30 seconds at 60° C., 1 minute at 72° C.) for 28 cycles, then 4 minutes of extension at 72° C. followed by 4° C.

The PCR products were visualized by electrophoresis. 7.5 μL of the 6× loading dye was added to each sample, mixed well and 35 μL of sample/dye mix was added to each lane on a 12.5% native acrylamide gel. The gel was run for 20 minutes at 100 volts followed by 1 hour 20 minutes at 400 volts, in 1× TBE buffer.

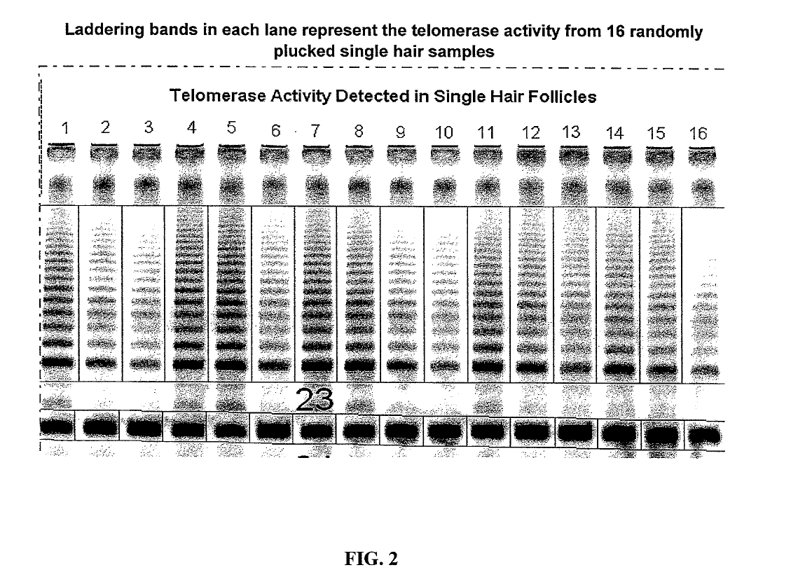

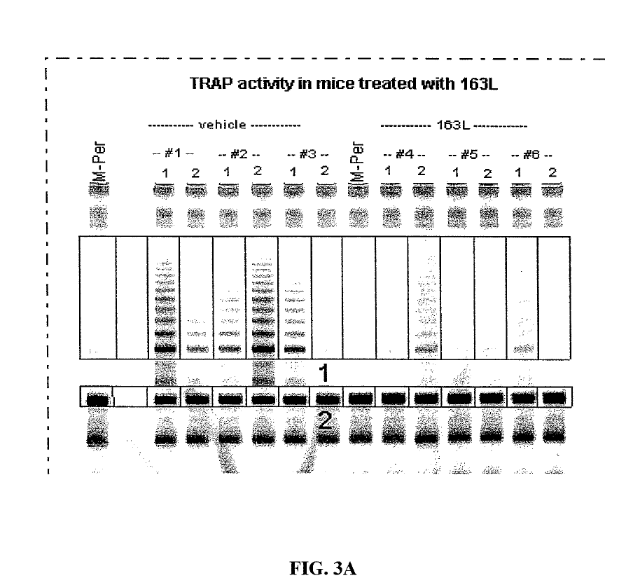

The acrylamide gel was scanned on a PhosphorImager using the red chemiluminexcence mode and the extension products quantitated. The hair follicle from healthy donor humans displayed robust telomerase activity. FIG. 2 is a photograph of the telomerase expression in human hair follicles from healthy donors.

### Example 5

### Telomerase Activity in Mice Treated With GRN163L

The GRN163L compound dissolved in saline was administered to mice by intraperitoneal injection at 50 mg/kg daily for 3 days. Control animals received saline only. Approximately 24 hours after the last injection, 6 whiskers from each animal were plucked (3 whiskers per pool). Three whiskers from each animal were lysed together in 30 μL of M-PER Lysis Buffer™ (Pierce Biotechnology, Illinois) by immersing the whiskers in the buffer and incubating the tube on ice for 5-6 hours with intermittent flicking.

5 μL of the hair follicle lysate was placed into the PCR plate (Stratgene, Calif.). 43.1 μL of PCR Mix-1 (see Example 4) was added to each well and mixed gently by pipetting. The PCR plate was sealed with adhesive film and the PCR was run for 30 minutes at 30° C.

After the telomere extension was completed, the temperature of the PCR machine was raised to 40° C. and 1.9 μL of PCR Mix-2 (see Example 4) was quickly added to each well. The PCR plate was resealed and the PCR machine was allowed to cycle (30 seconds at 94° C., 30 seconds at 60° C., 1 minute at 72° C.) for 28 cycles, then 4 minutes of extension at 72° C. followed by 4° C.

The PCR products were visualized by electrophoresis. 7.5 μL of the 6× loading dye was added to each sample, mixed well and 35 μL of sample/dye mix was added to each lane on a 12.5% native acrylamide gel. The gel was run for 20 minutes at 100 volts followed by 1 hour 20 minutes at 400 volts, in 1× TBE buffer. The acrylamide gel was scanned on PhosphorImager using the red chemiluminescence mode and the extension products quantitated.

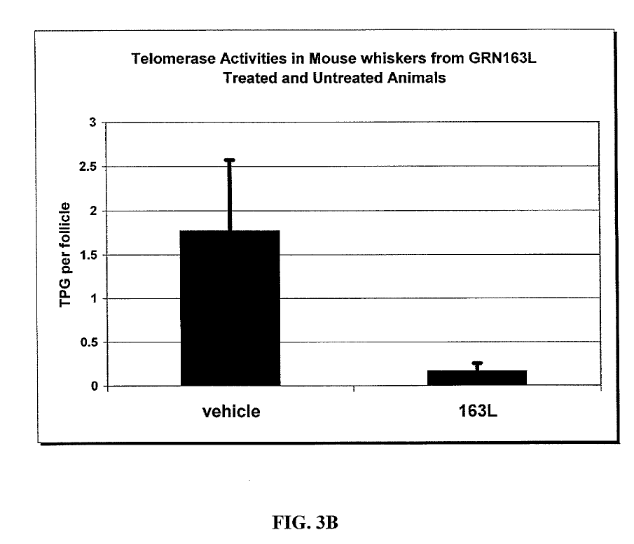

FIG. 3A shows the acrylamide gel of 3 mice who received 3 days of injections of vehicle or 63 L dissolved in vehicle. The intensity of the bands was measured and the value of total products generated (TPG) was calculated. TPG=total intensity of telomere ladder bands/total intensity of internal control band. FIG. 3B shows a graph of the TPG per follicle for vehicle and GRN163L treated mice. The whiskers of mice receiving vehicle had more TPG per follicle than the mice who received GRN163L in vehicle. When mice were treated with GRN163L an inhibition of telomerase activity was detected in vibrissa follicles.

Significant (>50%) inhibition of telomerase activity in mouse whisker cells was observed after animals were treated with GRN163L.

This confirms that mouse vibressa follicles can be used as a surrogate tissue for pharmacodynamic studies of telomerase-mediated therapies in vivo.

### Example 6

### Telomerase Activity in Rats Treated With GRN163L

A.) The GRN163L compound dissolved in saline was administered to rats by intraperitoneal injection at 50 mg/kg daily for 3 days. Control animals received saline only. Approximately 24 hours after the last injection, 6 whiskers from each animal were plucked (3 whiskers per pull). Four days after the last injection, 6 whiskers from each of another set of animals were plucked (3 whiskers per pull). Three whiskers from each animal were lysed together in 30 μL of M-PER Lysis Buffer™ (Pierce Biotechnology, Illinois) by immersing the whiskers in the buffer and incubating the tube on ice for 5-6 hours with intermittent flicking.

5 μL of the hair follicle lysate was placed into the PCR plate (Stratgene, Calif.). 43.1 μL of PCR Mix-1 was added to each well and mixed gently by pipetting. The PCR plate was sealed with adhesive film and the PCR was run for 30 minutes at 30° C.

After the telomere extension was completed, the temperature of the PCR machine was raised to 40° C. and 1.9 μL of PCR Mix-2 was quickly added to each well. The PCR plate was resealed and the PCR machine was allowed to cycle (30 seconds at 94° C., 30 seconds at 60° C., 1 minute at 72° C.) for 28 cycles, then 4 minutes of extension at 72° C. by 4° C.

The PCR products were visualized by electrophoresis. 7.5 μL of the 6× loading dye was added to each sample, mixed well and 35 μL of sample/dye mix was added to each lane on a 12.5% native acrylamide gel. The gel was run for 20 minutes at 100 volts followed by 1 hour 20 minutes at 400 volts, in 1× TBE buffer. The acrylamide gene was scanned on PhosphorImager using the red chemiluminescence mode and the extension products quantitated.

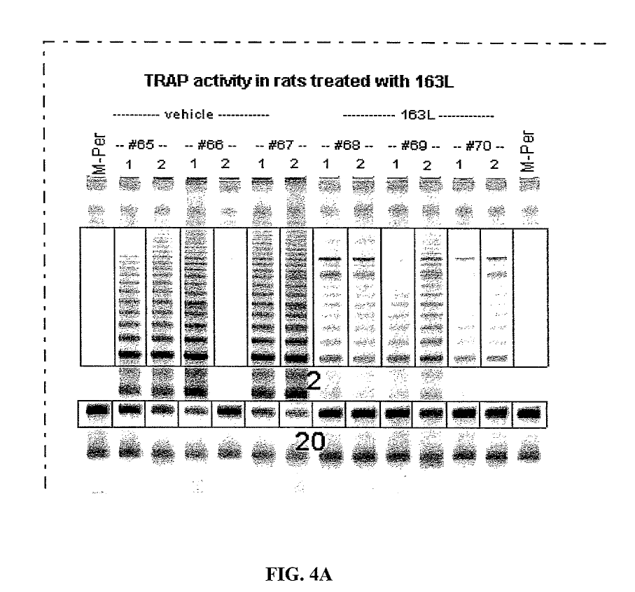

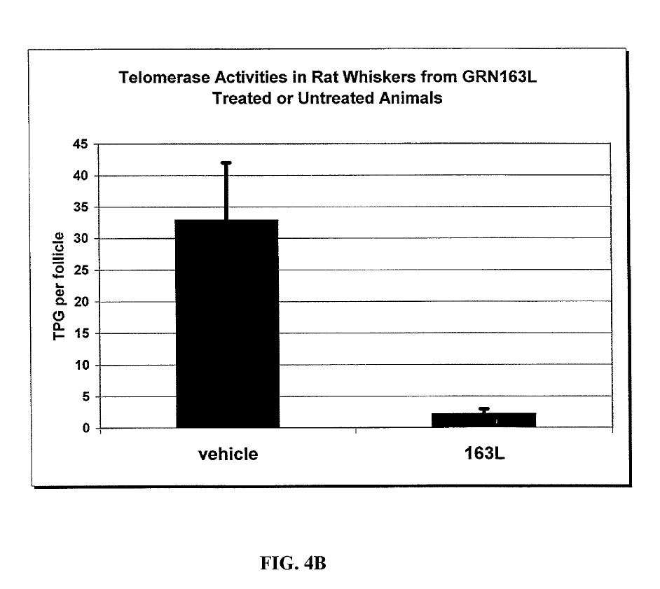

FIG. 4A shows the acrylamide gel of 3 rats who received 3 days of injections of vehicle or GRN163L dissolved in vehicle. FIG. 4B shows a graph of the TPG per follicle from vehicle and GRN163L treated animals. The whiskers of rats receiving vehicle had more TPG per follicle than the rats who received GRN163L in vehicle. Administration of GRN163L to rats substantially decreased the activity of the telomerase in whisker follicle cells.

B.) The GRN163L compound dissolved in saline at different concentrations was administered one time to rats. A mismatch control oligonucleotide, GRN140833 TAGGTGTAAGCAA (SEQ ID NO:3 1), was also administered to some animals. Approximately 24 hours after the injection, 6 whiskers from each animal were plucked (3 whiskers per pool). Three whiskers from each animal were lysed together in 30 μL of M-PER Lysis Buffer (Pierce Biotechnology, Illinois) by immersing the whiskers in the buffer and incubating the tube on ice for 5-6 hours with intermittent flicking.

5 μL of the hair follicle lysate was placed into the PCR plate (Stratgene, Calif.). 43.1 μL of PCR Mix-1 was added to each well and mixed gently by pipetting. The PCR plate was sealed with adhesive film and the PCR was run for 30 minutes at 30° C.

After the telomere extension was completed, the temperature of the PCR machine was raised to 40° C. and 1.9 μL of PCR Mix-2 was quickly added to each well. The PCR plate was resealed and the PCR machine was allowed to cycle (30 seconds at 94° C., 30 seconds at 60° C., 1 minute at 72° C.) for 28 cycles, then 4 minutes of extension at 72° C. followed by 4° C.

The PCR products were visualized by electrophoresis. 7.5 μL of the 6× loading dye was added to each sample, mixed well and 35 μL of sample/dye mix was added to each lane on a 12.5% native acrylamide gel. The gel was run for 20 minutes at 100 volts followed by 1 hour 20 minutes at 400 volts, in 1× TBE buffer. The acrylamide gene was scanned on PhosphorImager using the red chemiluminescence mode and the extension products quantitated.

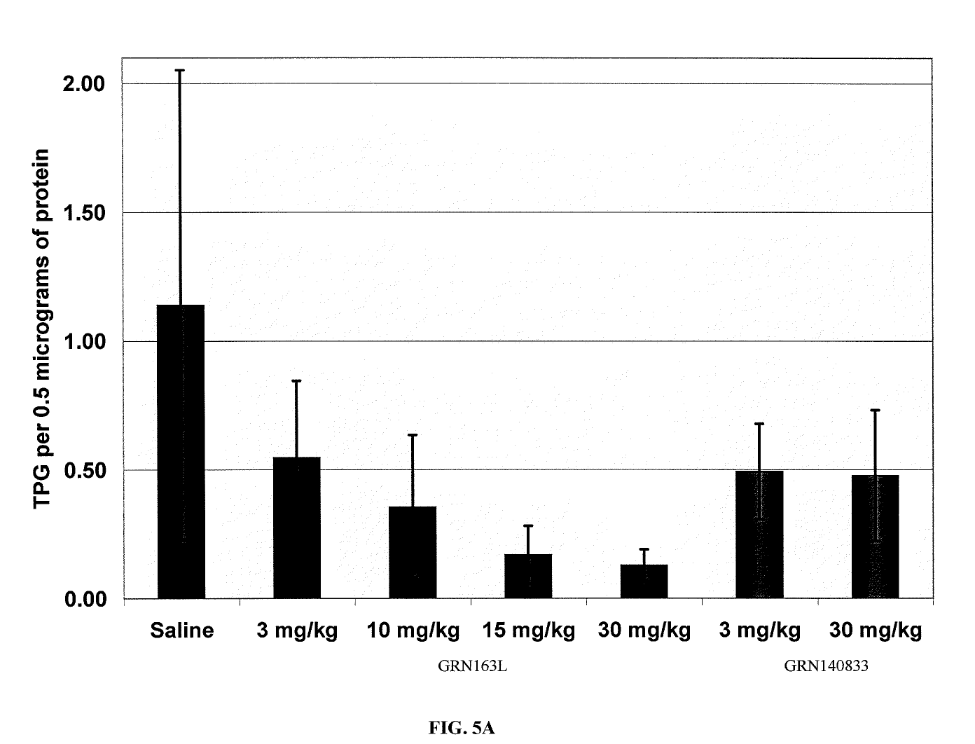

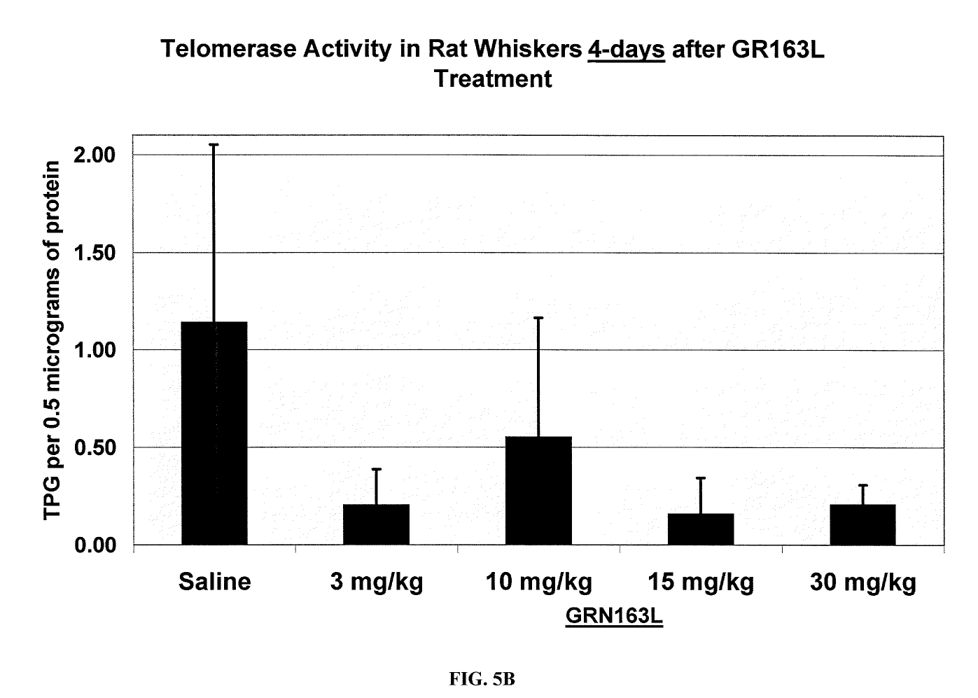

FIG. 5A is a graph showing the dose dependent and sequence specific inhibition of telomerase activity in rat vibressa hair follicles 1 day after treatment with GRN163L. FIG. 5B is a graph showing the dose dependent and sequence specific inhibition of telomerase activity in rat vibressa hair follicles 4 days after treatment with GRN163 L. When rats were treated with GRN163 L, a dose dependent inhibition of telomerase activity was detected in vibrissa hair follicle cells. In FIGS. 5A and 5B the value of TPG is given per set amount of total protein obtained from the follicles.

This confirms that rat vibressa follicles can be used as a surrogate tissue for pharmacodynamic studies of telomerase-mediated therapies in vivo.

### Example 7

### Telomerase Activity in Human Hair Follicles Treated With GRN163L in Vitro

Human hair was plucked from healthy donors as described in Example 2. Three hair follicles were placed in a test tube and a solution of GRN163L in CCM (RPMI1640 and 10% fetal bovine serum) at different concentrations was added to the hair. The hair follicles were incubated in GRN163L containing CCM at room temperature for 2 hours. The hair follicles were removed from the GRN163L solution and gently washed 2 times in 1 mL phosphate buffered saline. Hair follicles were then placed into the Lysis Buffer and the level of telomerase activity was determined as described in Example 4.

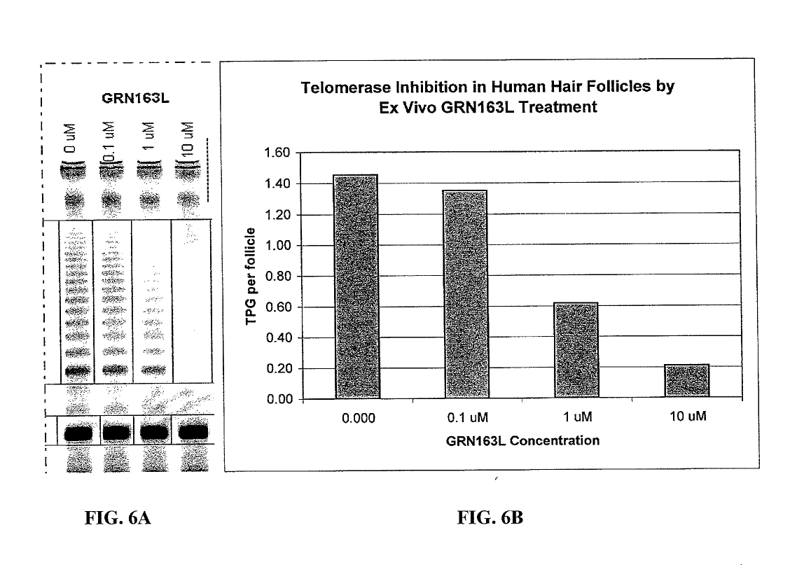

FIG. 6A is a photograph of the acrylamide gel from human hair follicles which were treated with 0.1 μM, 1 μM, or 10 μM of GRN163 or vehicle alone. FIG. 6B shows a graph of the TPG per treatment group. The human hair follicles treated with GRN163L in vitro showed a dose dependent decrease in telomerase activity. This confirms that human hair follicles can be used as a surrogate tissue for pharmacodynamic studies of telomerase-mediated therapies in vivo.

### Example 8

### Telomerase Inhibition in Mouse Follicle Cells Correlates with Xenograft Tumor Growth Inhibition After 4 Weeks of GRN163L Treatment

Mice were injected with A549 human lung carcinoma cells (ATCC, Virginia) (3 million cells/animal) subcutaneously on the back. There were 10 animals per treatment group.

24 hours after the cell injection, the GRN163L compound dissolved in saline was administered to mice by intraperitoneal injection at 30 mg/kg. Control animals received saline only. The GRN163L compound was administered three times per week (tiw) for the duration of the study and the size of the tumor mass was measured.

At the end of the study, hair follicles from 3 animals from each treatment group were plucked and the level of telomerase activity measured. The three animals chosen for each group represented the animals with the largest, the median and the smallest tumor volume within the group.

The hair follicles were placed into the Lysis Buffer and the level of telomerase activity was determined as described in Example 4.

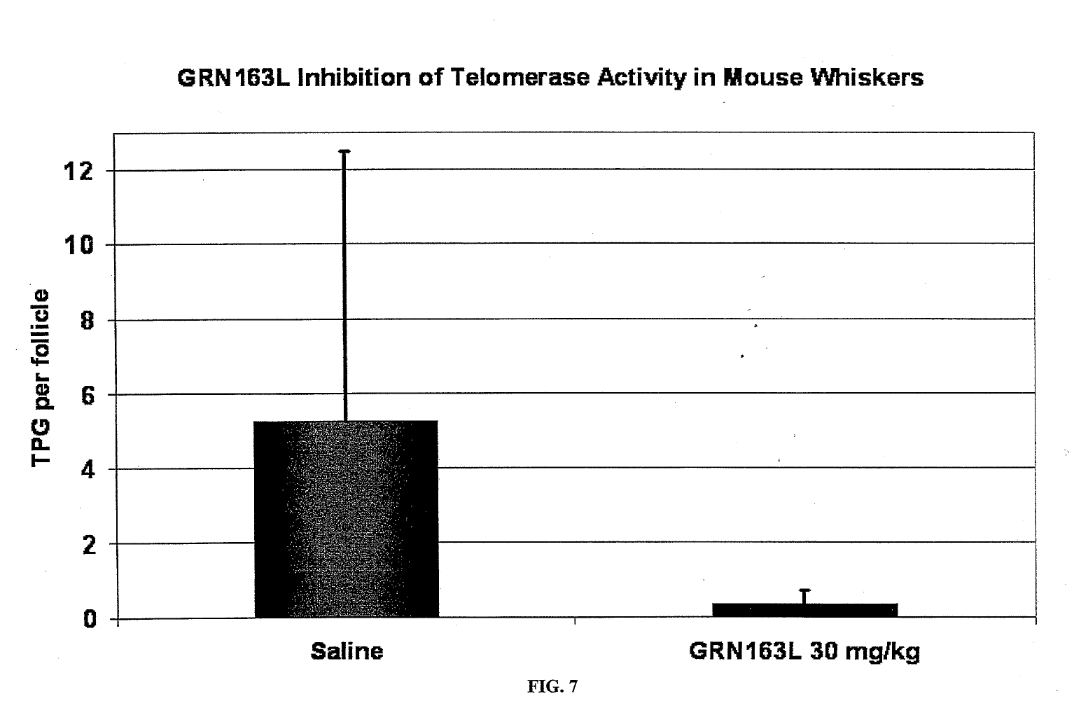

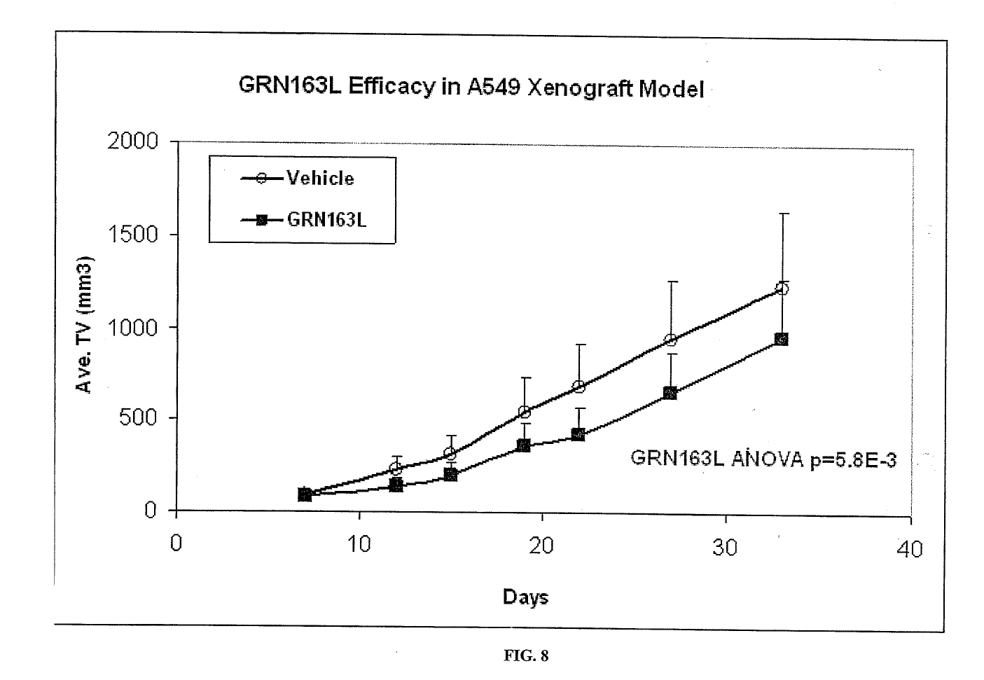

FIG. 7 is a graph of the TPG per group in mouse whisker follicles after 4 weeks of treatment with GRN163L. FIG. 8 is a graph of the average tumor volumes in the mice after treatment with GRN163L for a period of 4 weeks. Animals receiving GRN163L showed reduced tumor growth over the 4 week period. The inhibition of telomerase activity in mouse vibrissa follicles correlates with the reduced tumor volume observed.

### Example 9

### Gel TRAP Assay for Hair Follicles

This experiment was to quantitatively evaluate telomerase activity from clinical and preclinical samples. A normalization method was developed.

Hair follicle samples were collected as a single hair follicle or a group of hair follicles (such as 10 hair follicles) freshly or put into freezing medium and frozen at 80° C. until ready to test. Each single hair follicle was lysed in 60μL of M-Per lysis buffer (Pierce Biotechnology, Inc., Rockford Ill.) on ice for about 6 hrs. The lysed hair follicle solution was centrifuged at 14K (bench top mini centrifuge) at 4° C. for 20 min. The hair follicle extract was transferred to a fresh tube and the extracts were ready for performing the TRAP analysis for telomerase activity.

1-step TRAP reaction master mix preparation:

| | stock con. | per rxn, uL | final con. |
|---|---|---|---|
| H2O | | 37.5 | |
| 10x TRAP buffer | | 5 | 1x |
| w/ BSA | | | |
| dNTP | 2.5 mM | 1 | 50 μM |
| Cy5-TS | 0.5 mg/mL, 83 μM | 0.1 | 1 ng/μL |
| ACX | 0.1 mg/mL, 11 μM | 1 | 2 ng/μL |
| Taq polymerase | 5 U/μL | 0.4 | 0.04 U/μL |

The TRAP reaction master mix 40-45 μL/tube was mixed with 5-10 μL of hair extract.

A PCR reaction was run as follows: 30° C. for 30 minutes; repeat 28 cycles of the following 3-step reaction: 94° C. for 30 seconds; 60° C. for 30 seconds; 72° C. for 1 minute; 72° C. for 4 minutes; hold at 4° C. The dynamic range of the test procedure was defined by running a serial dilution of 293 cell extract, a telomerase positive cell line.

The PCR products were visualized by electrophoresis. 7.5 μL of the 6× loading dye is added to each sample, mixed well and 35 μL of sample/dye mix is added to each lane on a 12.5% native acrylamide gel. The gel was run for 20 minutes at 100 volts followed by 1 hour 20 minutes at 400 volts, in 1× TBE buffer. The acrylamide gene was scanned on PhosphorImager using the red chemiluminescence mode and all of the extension products quantitated. The telomerase activity was expressed by Pixel volume (signal above background). This procedure results in higher sensitivity than the two step procedure.

The hair follicle sample is very heterogonous in size and biological stages. To adjust for this the hair follicle sample is normalized.

Total RNA from cytosol contains about 80% of rRNA and it reflects the cell number from which the extract was made. Telomerase reverse transcriptase co-purifies or co-extracts with ribosomal RNP which is a major source of rRNA. Measuring total RNA thus can reflect the cell number and extraction efficiency of the hair follicle samples. Therefore using total RNA information to normalize the assay result enables the assay to be more quantitave and accurate.

The following procedure was used to determine total RNA concentrations in solution such as M-PER extract of hair follicles or other tissue types using the RIBOGREEN® Quantification Kit (Molecular Probes cat. #R-11490, Eugene Oreg.). RIBOGREEN® is an ultra-sensitive fluorescent dye that is used in the detection and quantification of nucleic acids. In its free form, RIBOGREEN® exhibits little fluorescence and possesses a negligible absorbance signature. When bound to nucleic acids, the dye fluoresces with an intensity that is several orders of magnitude greater than the unbound form. The fluorescent signal can be detected at excitation ˜480 nm and the emission ˜525 nm. Using a fluorescence microplate reader, as little as 1 ng/mL RNA can be detected. The presence of protein in the sample to be tested does not affect the absorbance, and thus allows quantifying total RNA (some DNA if no DNase treatment) from soluble protein extract from human or animal samples.

The hair follicle extract was diluted in TE buffer 100-250 fold (depends on the HF size, 1-5 uL extract) and 100 uL of the diluted extract was placed in a well in 96-well plate (3×). RIBOGREEN® dye 1:2000 dilution was added to the well and the flourescence of the well was read. If a hair follicle generates a TRAP signal of 5e6 pixel and the RNA concentration in the same extract is 250 ng/mL, the normalized TRAP signal is 2e4. The RNA number reflects the cell numbers of the hair follicles. The total RNA analysis allows the hair follicle measurement to be more quantitative and accurate.

Although the invention has been described with respect to particular embodiments and applications, those skilled in the art will appreciate the range of applications and methods of the invention disclosed herein.

1. A method for measuring telomerase activity in a subject exposed to a telomerase modulator, comprising measuring the level of telomerase activity in a hair follicle cell of the subject.

2. A method of measuring the response of a subject to a telomerase modulator, comprising measuring the level of telomerase activity in a hair follicle cell of the subject, wherein a difference in the level of telomerase activity in the hair follicle cell after exposure of the subject to the telomerase modulator as compared to the level of activity in a hair follicle cell without telomerase modulator indicates a response.

3. A method for evaluating the biological response of a subject to a telomerase modulator comprising the steps of:
(a) exposing a subject to the telomerase modulator
(b) obtaining a hair follicle from said subject;
(c) determining the telomerase activity in the hair follicle of the subject,
wherein a difference in the level of telomerase activity in a hair follicle after exposure of the subject to the telomerase modulator as compared to the level of activity in a hair follicle without telomerase modulator indicates a response.

4. The method of claim 1, wherein the hair follicle is obtained from a human.

5. The method of claim 1, wherein the hair follicle is obtained by plucking a hair.

6. The method of claim 1, wherein the hair follicle is stained with a DNA binding label and hair follicles having live cells in the sleeve region are selected for measurement of telomerase activity.

7. The method of claim 1, wherein the method of measuring telomerase activity comprises
a) preparing a cell extract from said hair follicle
b) measuring the amount of nucleotides incorporated into elongation products by the telomerase isolated in step (a).

8. The method of claim 1, wherein said method comprising the steps of:
(a) preparing a cell extract from said hair follicle;
(b) placing an aliquot of said cell extract in a reaction mixture comprising a telomerase substrate lacking a telomeric repeat sequence and a buffer in which telomerase can catalyze extension of said telomerase substrate by addition of telomeric repeat sequences;
(c) adding to said reaction mixture a primer comprising a sequence sufficiently complementary to a telomeric repeat to hybridize specifically thereto under conditions such that if an extended telomerase substrate is present in said reaction mixture, said primer will hybridize to said extended telomerase substrate and extend to form a complementary copy of said extended telomerase substrate, thereby forming duplex DNA molecules comprising an extended telomerase substrate bound to an extended primer; and
(d) correlating presence of telomerase activity in said cell sample with presence of duplex DNA molecules comprising an extended telomerase substrate bound to an extended primer and absence of telomerase activity in said cell sample with absence of said duplex DNA molecules.

9. The method of claim 8, wherein step (c) additionally comprises steps of:
(a) heating said reaction mixture to denature said duplex DNA molecules; and
(b) cooling said reaction mixture to a temperature at which complementary nucleic acids can hybridize and said primer can extend if extended telomerase substrates are present.

10. The method of claim 9, wherein said heating and cooling steps are repeated at least 5 times, and said primer is present in amounts sufficient for the formation of extended primers during each cooling step.

11. The method of claim 8, wherein a template-dependent DNA polymerase is present in the reaction mixture of step (c) and said primer is extended by addition of nucleotides to said primer by said DNA polymerase.

12. The method of claim 8, wherein a template-dependent DNA ligase is present in the reaction mixture of step (c) and said primer is extended by ligation of an oligonucleotide ligomer to said primer by said DNA ligase.

13. The method of claim 11, wherein the template-dependent DNA polymerase is a thermostable template-dependent DNA polymerase.

14. The method of claim 12, wherein the template-dependent DNA ligase is a thermostable template-dependent DNA ligase.

15. The method of claim 8, wherein said reaction mixture comprises a labelled telomerase substrate.

16. The method of claim 8, wherein said reaction mixture comprises a labelled primer.

17. The method of claim 16, wherein said label is selected from the group consisting of a radioactive molecule, a fluorescent molecule, a phosphorescent molecule, a ligand for a receptor, biotin, and avidin.

18. The method of claim 8, wherein said telomerase substrate lacking a telomeric repeat sequence is 5′-AATCCGTCGAGCAGAGTT-3′ (SEQ ID NO:31).

19. The method of claim 8, wherein said primer comprises a non-telomeric repeat sequence at a 5′-end of said primer.

20. The method of claim 19, wherein said primer is
(SEQ ID NO:27)
5′-CCCTTACCCTTACCCTTACCCTAA-3′,
(SEQ ID NO:32)
5′-GCGCGGCTAACCCTAACCCTAACC-3′
or
(SEQ ID NO:29)
5′-GCGCGGCTTACCCTTACCCTTACCCTAACC-3′.

21. The method of claim 8, wherein the presence of extended telomerase substrate is detected using a branched DNA probe.

22. The method of claim 1, further comprising normalizing the level of telomerase activity in the hair follicle cell relative to the amount of RNA in the hair follicle cell.

23. The method of claim 1, wherein the telomerase modulator is telomerase inhibitor comprising an oligonucleotide sequence effective to bind by sequence-specific hybridization to a template region of hTR.

24. The method of claim 23 wherein the oligonucleotide sequence comprises internucleoside linkages selected from N3′→P5′ phosphoramidate and N3′→P5′ thiophosphoramidate linkages.

25. The method of claim 24 wherein the telomerase inhibitor comprises a lipid moiety (i) selected from the group consisting of fatty acids, sterols, and derivatives thereof, and (ii) attached covalently to one end of the oligonucleotide.

26. The method of claim 24 wherein the oligonucleotide is characterized by:
N3′→P5′ thiophosphoramidate internucleoside linkages;
having the sequence identified as SEQ ID NO: 12; and
a palmitoyl (C16) moiety linked to the 5′ terminus of the oligonucleotide via a glycerol or aminoglycerol linker.

27. The method of claim 26 wherein the telomerase inhibitor is the compound designated herein as GRN163L.

28. The method of claim 1, wherein the telomerase modulator is an inhibitor which is administered in an amount effective to inhibit the proliferation of cancer cells in the subject, when the telomerase modulator is administered alone.

29. The method of claim 27, wherein said exposure of the telomerase inhibitor includes infusing the oligonucleotide telomerase inhibitor intravenously into the subject, under infusion conditions effective to produce a blood concentration of the inhibitor of between 1 nM and 100 μM.

30. The method of claim 1, wherein the subject is exposed to the telomerase modulator for the treatment of cancer and the cancer is selected from the group consisting of breast cancer, ovarian cancer, basal-cell carcinoma, small-cell lung carcinoma, non-small cell lung carcinoma, squamous cell carcinoma, hepatocellular carcinoma, renal cell carcinoma, and multiple myeloma.

31. A method for evaluating the biological response of a subject exposed to a telomerase modulator comprising the steps of:
(a) obtaining a hair follicle from said subject;
(b) preparing a cell extract from the hair follicle by lysing the cells;
(c) incubating said cell extract in a reaction mixture comprising an exogenous telomerase substrate under conditions such that telomerase can catalyze extension of said telomerase substrate by addition of telomeric repeat sequences;
(d) replicating said extended telomerase substrate; and
(e) correlating presence of telomerase activity in said cell extract with presence of said extended telomerase substrate and absence of telomerase activity in said cell extract with absence of said extended telomerase substrate.

32. A method for evaluating the biological response of a subject exposed to a telomerase modulator comprising the steps of:
(a) obtaining a hair follicle from said subject;
(b) preparing a cell extract from the hair follicle by lysing the cells;
(c) incubating the cell extract in a reaction mixture comprising a telomerase substrate and a buffer in which telomerase can catalyze extension of said telomerase substrate by addition of telomeric repeat sequences;
(d) adding to said reaction mixture a template-dependent RNA polymerase that recognizes a promoter sequence operably linked to said telomerase substrate;
(e) allowing said RNA polymerase to form an RNA copy of said extended telomerase substrate if an extended telomerase substrate is present in said reaction mixture; and
(f) correlating presence of telomerase activity in said cell extract with presence of RNA copies of said extended telomerase substrate and absence of telomerase activity in said cell extract with absence of said RNA copies.

33. The method of claim 32, wherein said RNA polymerase recognizes a single-stranded nucleic acid substrate.

34. The method of claim 32, wherein said RNA polymerase is selected from the group consisting of an N4 RNA polymerase, an SP6 RNA polymerase, a T7 RNA polymerase and a T3 RNA polymerase.

35. A kit for measuring telomerase activity in a subject exposed to a telomerase modulator, said kit comprising:
(a) tweezers for plucking hair;
(b) a lysis buffer;
(c) a telomerase substrate; and
(d) a primer comprising a sequence complementary to a telomeric repeat sequence.

36. The kit of claim 35 wherein said kit further comprises an assay buffer.

37. The kit of claim 35, wherein said kit further comprises an oligonucleotide control for primer extension.

38. The kit of claim 35, wherein said telomerase substrate is 5′-AATCCGTCGAGCAGAGTT-3′ (SEQ ID NO:3 1), said primer is 5′-GCGCGGCTTA CCCTTACCCTTACCCTAACC-3′ (SEQ ID NO:32) and said kit further comprises 10× TRAP reaction buffer, a stock solution of dATP, dGTP, dCTP and dTTP, 5′-AATCCGTCGAGCAGAGTTAAAAGGCCGAGAAGCGAT-3′ (SEQ ID NO:33) and 5′-ATCGCTTCTCGGCCTTTT-3′ (SEQ ID NO:29).

39. A method of selecting hair follicles with live cells comprising staining the hair follicle with a DNA binding label.

40. The method of claim 39 wherein the DNA binding label is 4′,6-diamidino-2-phenylindole (DAPI).
